SEQUENCE LISTING (1 sequence)

SEQ ID NO: 1 - protein, 8 aa, Artificial Sequence
  OVA epitope polypeptide
SIINFEKL

(19) United States
(12) Patent Application Publication
(10) Pub. No.: US 2023/0145599 A1
(43) Pub. Date: May 11, 2023

(54) Microneedle Array Delivery of Adenovirus Vectored Vaccines With and Without Adjuvants

(71) Applicant: University of Pittsburgh - Of the Commonwelth System of Higher Education, Pittsburgh, PA (US)

(72) Inventors: Louis Falo, Jr., Wexford, PA (US); Andrea A. Gambotto, Pittsburgh, PA (US); Geza Erdos, Wexford, PA (US)

(21) Appl. No.: 17/917,873

(22) PCT Filed: Apr. 8, 2021
(86) PCT No.: PCT/US2021/026364

§ 371 Date: Oct. 7, 2022

Related U.S. Application Data
(60) Provisional application No. 63/007,426, filed on Apr. 9, 2020.

(51) Int. Cl. A61M 37/00; C12N 15/86; A61K 39/235

(52) U.S. Cl. CPC A61M 37/0015; C12N 15/86; A61K 39/235; A61M 2037/0053; A61M 2037/0023; A61M 2037/0061; A61K 2039/53

(57) ABSTRACT

Provided herein are microneedle array devices for delivery of recombinant adenovirus particles, methods of making the devices, and used for the devices. The microneedle array devices are storage-stable at 4° C., retaining adenovirus infectivity for at least one month.

### CROSS REFERENCE TO RELATED APPLICATIONS

This application is the United States national phase of International Application No. PCT/US2021/026364 filed Apr. 8, 2021, and claims priority to U.S. Provisional Patent Application No. 63/007,426 filed Apr. 9, 2020, the disclosures of which are hereby incorporated by reference in their entirety.

### STATEMENT REGARDING FEDERAL FUNDING

This invention was made with government support under Grant Nos. AR074285, AR071277, and AR068249, awarded by the National Institutes of Health. The government has certain rights in the invention.

Adenoviral vectors encoding therapeutic agents are a promising. Delivery of polypeptides and functional RNA gene products can be achieved with broad target cell tropism. The technical field relating to development of adenoviral vectors and preparation of adenoviral infectious particles is mature, with a significant number of commercial entities providing specific adenoviral vector products, adenovirus cloning systems, and/or customized adenovirus products and vector development. Preparation of adenoviral vectors expressing therapeutic polypeptides, immunogens, gene editing machinery, and RNA interference (RNAi) reagents is generally routine using standard genetic techniques and established techniques relating to development and production replicative and non-replicative adenoviral particles.

Genetic immunization based on recombinant DNA technology is an attractive approach for induction of robust antiviral or antitumor immunity. Specifically, adenoviral vaccines encoding target antigen transgenes are the subject of extensive preclinical studies due to their established capacity for generating immune responses. The skin is an ideal vaccination site containing an innate immune network that is exquisitely responsive to environmental stimuli and capable of inducing a pro-inflammatory microenvironment, favoring the generation of strong and long-lasting adaptive immunity. To exploit the readily accessible cutaneous immune network, vaccine delivery technologies, such as microneedle arrays (MNAs), have been developed to precisely and reproducibly target immunologically active cargos to skin microenvironments.

Despite the therapeutic promise of recombinant adenoviral particles, stability of such particles is such that the production, storage, distribution, and use of such vectors is limited. There is a substantial need for stable compositions and products for storage and delivery of recombinant adenovirus particles.

### SUMMARY OF THE INVENTION

A microneedle device is provide. The device comprises: a backing layer; and a plurality of microneedles extending from the backing layer, and comprising a dissolvable (aqueous-soluble) and/or bioerodible matrix comprising trehalose, and a recombinant adenovirus particle comprising a gene for expressing a polypeptide or RNA.

A method of eliciting a therapeutic effect in a patient is provided. The method comprises placing the microneedle device as described above on the skin of the patient to cause the plurality of microneedles to enter the skin of the patient, thereby eliciting the therapeutic effect in the patient.

A method of expressing a transgene in a patient also is provided, comprising placing the microneedle device as described above on the skin of the patient to cause the plurality of microneedles to enter the skin of the patient, thereby introducing the adenovirus into a cell of the patient for expression of the gene encoded by the adenovirus.

A method of forming a microneedle device is provided. The method comprises: forming or providing a production mold of a flexible material, the production mold comprising a plurality of cavities that are shaped to define a plurality of respective microneedles having a stem, a head, a filleted base, and at least one undercut feature, the microneedles optionally having a length of 1 mm or less; delivering a first dissolvable or bioerodible material comprising trehalose into at least the microneedle head portion defined by the respective cavities of the production mold, and prior to or during delivery of the first dissolvable or bioerodible material into at least the microneedle head portion, incorporating a recombinant adenovirus particle comprising a gene for expressing a polypeptide or RNA into the first dissolvable or bioerodible material to produce a dissolvable or biodegradable matrix; delivering the first dissolvable or bioerodible material and/or one or more additional dissolvable or bioerodible materials into the cavity and forming a plurality of microneedles in the production mold that include the dissolvable or biodegradable matrix; and removing the microneedles from the production mold by pulling the microneedles out of the mold, wherein the flexible material of the production mold has sufficient elasticity to allow for the molded microneedle array to be removed from the production mold, e.g., in a single pull, without damaging the integrity of the shape of the microneedles as defined by the mold.

According to a first embodiment or aspect, a microneedle device is provided, comprising:
a backing layer; and
a plurality of microneedles extending from the backing layer, and comprising a dissolvable (aqueous-soluble) and/or bioerodible matrix comprising trehalose, and a recombinant adenovirus particle comprising a gene for expressing a polypeptide or RNA.

According to a second embodiment or aspect, provided is the microneedle device of the first embodiment or aspect, wherein the recombinant adenovirus in the microneedle device retains viability for at least one month at 4° C.

According to a third embodiment or aspect, provided is the microneedle device of the first embodiment or aspect, wherein the number of infectious units (IU) of the recombinant adenovirus in the microneedle decreases by less than 50%, 40%, 30%, or 25% on storage for one month at 4° C.

According to a fourth embodiment or aspect, provided is the microneedle device of the first embodiment or aspect, wherein the gene expresses a polypeptide.

According to a fifth embodiment or aspect, provided is the microneedle device of the fourth embodiment or aspect, wherein the polypeptide is an immunogen.

According to a sixth embodiment or aspect, provided is the microneedle device of the fifth embodiment or aspect, wherein the microneedle further comprise a compound or composition having immune stimulant or adjuvant effect.

According to a seventh embodiment or aspect provided is the microneedle device of the sixth embodiment or aspect, wherein the compound or composition having immune stimulant or adjuvant effect is a double-stranded RNA, or an analog or derivative thereof.

According to an eighth embodiment or aspect, provided is the microneedle device of the sixth embodiment or aspect, wherein the compound or composition having immune stimulant or adjuvant effect is a TLR3 agonist.

According to a ninth embodiment or aspect, provided is the microneedle device of the sixth embodiment or aspect, wherein the compound or composition having immune stimulant or adjuvant effect is Poly(I:C) or Poly-ICLC.

According to a tenth embodiment or aspect, provided is the microneedle device of the sixth embodiment or aspect, wherein the compound or composition having immune stimulant or adjuvant effect is a TLR4 agonist; a TLR5 agonist; a TLR 7/8 agonist; a TLR9 agonist; a Stimulator of Interferon Genes (STING) pathway agonist; a stimulatory neuroimmune mediator; a neurokinin 1 (NK1) receptor agonist; a saponin related adjuvant; a purinoergic receptor agonist; or an oil-in-water emulsion adjuvant.

According to an eleventh embodiment or aspect, provided is the microneedle device of the tenth embodiment or aspect, wherein the compound or composition having immune stimulant or adjuvant effect is an LPS, monophosphoryl lipid derivative, a flagellin derivative, imiquimod, R848, a CpG nucleic acid sequence, ADU-S100, a calcitonin gene-related peptide (CGRP), Hemokinin 1, Substance P, QS-21 (Quillaja saponaria), ATP, or MF59.

According to a twelfth embodiment or aspect, provided is the microneedle device of the first embodiment or aspect, wherein the gene expresses an antisense RNA or an RNAi reagent.

According to a thirteenth embodiment or aspect, provided is the microneedle device of the twelfth embodiment or aspect, wherein the gene expresses an shRNA.

According to a fourteenth embodiment or aspect, provided is the microneedle device of any one of the first through the thirteenth embodiment or aspect, wherein the plurality of microneedles have a shape that comprises a first cross-sectional dimension at a head portion distal to the backing layer, a second cross-sectional dimension at a stem portion proximal to the backing layer, and a third cross-sectional dimension at an intermediate portion located between the top portion and the bottom portion having a cross-sectional dimension greater than the first and second cross-sectional dimensions.

According to a fifteenth embodiment or aspect, provided is the microneedle device of the fourteenth embodiment or aspect, wherein the plurality of microneedles comprise a stem portion adjacent or proximal to the backing layer, and a head portion attached to the stem portion distal to the backing layer, the microneedles having a barbed or undercut profile in which the head portion having a cross section adjacent to the stem is larger than a cross section of the stem adjacent to the head.

According to a sixteenth embodiment or aspect, provided is the microneedle device of the fourteenth or the fifteenth embodiment or aspect, wherein a plurality of microneedles comprise a plurality of layers of one or more dissolvable or bioerodible, biocompatible material.

According to a seventeenth embodiment or aspect, provided is the microneedle device of any one of the first through the sixteenth embodiment or aspect, wherein the plurality of microneedles further comprise carboxymethyl cellulose.

According to an eighteenth embodiment or aspect, provided is the microneedle device of any one of the first through the seventeenth embodiment or aspect, wherein the plurality of microneedles further comprise, polyvinylpyrrolidone, maltodextrin, silk, hyaluronic acid, poly(lactic-co-glycolic acid), poly(lactic acid), poly(vinyl alcohol), polyethylene glycol, or a combination of any two or more of the preceding.

According to a nineteenth embodiment or aspect, provided is the microneedle device of any one of the first through the eighteenth embodiment or aspect, wherein the plurality microneedles have a layered structure with the adenovirus particles located substantially at the tip of the microneedles.

According to a twentieth embodiment or aspect, provided is the microneedle device of any one of the first through the nineteenth embodiment or aspect, wherein the plurality of microneedles each have a length of 1 mm or less.

According to a twenty-first embodiment or aspect, provided is the microneedle device of any one of the first through the twentieth embodiment or aspect, wherein the plurality of microneedles have a filleted base.

According to a twenty-second embodiment or aspect, provided is the microneedle device of any one of the first through the twenty-first embodiment or aspect, wherein the plurality of microneedles comprise a stem, a head, a filleted base, and at least one undercut feature.

According to a twenty-third embodiment or aspect, provided is the microneedle device of the twenty-second embodiment or aspect, wherein the plurality of microneedles comprise the recombinant adenovirus particles located substantially in the head of the microneedles.

According to a twenty-fourth embodiment or aspect, provided is the microneedle device of the twenty-second or twenty-third embodiment or aspect, wherein the at least one undercut feature is directly below the microneedle head.

According to a twenty-fifth embodiment or aspect, provided is the microneedle device of any one of the twenty-second through the twenty-fourth embodiment or aspect, wherein the stem is formed from a first dissolvable or bioerodible material.

According to a twenty-sixth embodiment or aspect, provided is the microneedle device of the twenty-fifth embodiment or aspect, wherein the dissolvable or bioerodible material of the stem comprises carboxymethyl cellulose, trehalose, polyvinylpyrrolidone, maltodextrin, silk, hyaluronic acid, poly(lactic-co-glycolic acid), poly(lactic acid), poly(vinyl alcohol), polyethylene glycol, or a combination of any two or more of the preceding.

According to a twenty-seventh embodiment or aspect, provided is the microneedle device of any one of the twenty-second through the twenty-fourth embodiment or aspect, wherein the stem is formed from a non-dissolvable material.

According to a twenty-eighth embodiment or aspect, provided is the microneedle device of any one of the twenty-second through the twenty-seventh embodiment or aspect, further comprising a dissolvable material different from the first dissolvable or bioerodible material at a portion of the stem, such as adjacent to the microneedle head.

According to a twenty-ninth embodiment or aspect, provided is the microneedle device of the twenty-eighth embodiment or aspect, wherein the dissolvable material different from the first dissolvable or bioerodible material dissolves more quickly than the dissolvable and/or bioerodible matrix comprising the recombinant adenovirus particle.

According to a thirtieth embodiment or aspect, provided is the microneedle device of the twenty-eighth or twenty-ninth embodiment or aspect, wherein the dissolvable material different from the first dissolvable or bioerodible material comprises a low molecular weight compound or composition that rapidly dissolves in the microneedle in water, e.g., dissolves in less than 30 seconds, less than 20 seconds, less than 10 seconds, or less than 5 seconds in water.

According to a thirty-first embodiment or aspect, provided is the microneedle device of any one of the twenty-eighth through the thirtieth embodiment or aspect, wherein the dissolvable material different from the first dissolvable or bioerodible material comprises glucose, trehalose, sucrose, maltodextrin, polyvinylpyrrolidone, or a combination of two or more of any of the preceding.

According to a thirty-second embodiment or aspect, provided is the microneedle device of any one of the first through the thirty-first embodiment or aspect, wherein the Adenovirus is an Ad5 vector for expression of a transgene.

According to a thirty-third embodiment or aspect, provided is a method of eliciting a therapeutic effect in a patient, comprising placing the microneedle device of any one of the first through the thirty-second embodiment or aspect, on the skin of the patient to cause the plurality of microneedles to enter the skin of the patient, thereby eliciting the therapeutic effect in the patient.

According to a thirty-fourth embodiment or aspect, provided is a method of expressing a transgene in a patient, comprising placing the microneedle device of any one of the first through the thirty-second embodiment or aspect, on the skin of the patient to cause the plurality of microneedles to enter the skin of the patient, thereby introducing the adenovirus into a cell of the patient for expression of the gene encoded by the adenovirus.

According to a thirty-fifth embodiment or aspect, provided is a method of forming a microneedle device, comprising:
forming or providing a production mold of a flexible material, the production mold comprising a plurality of cavities that are shaped to define a plurality of respective microneedles having a stem, a head, a filleted base, and at least one undercut feature, the microneedles optionally having a length of 1 mm or less;
delivering a first dissolvable or bioerodible material comprising trehalose into at least the microneedle head portion defined by the respective cavities of the production mold, and prior to or during delivery of the first dissolvable or bioerodible material into at least the microneedle head portion, incorporating a recombinant adenovirus particle comprising a gene for expressing a polypeptide or RNA into the first dissolvable or bioerodible material to produce a dissolvable or biodegradable matrix;
delivering the first dissolvable or bioerodible material and/or one or more additional dissolvable or bioerodible materials into the cavity and forming a plurality of microneedles in the production mold that include the dissolvable or biodegradable matrix; and
removing the microneedles from the production mold by pulling the microneedles out of the mold,
   wherein the flexible material of the production mold has sufficient elasticity to allow for the molded microneedle array to be removed from the production mold, e.g., in a single pull, without damaging the integrity of the shape of the microneedles as defined by the mold.

According to a thirty-sixth embodiment or aspect, provided is the method of the thirty-fifth embodiment or aspect, wherein the production mold defines at least one undercut feature in the microneedles directly below a microneedle head.

According to a thirty-seventy embodiment or aspect, provided is the method of the thirty-fifth or thirty-sixth embodiment or aspect, wherein the stem is formed from the first dissolvable or bioerodible material.

According to a thirty-eighth embodiment or aspect, provided is the method of any one of the thirty-fifth through the thirty-seventh embodiment or aspect, wherein the first dissolvable or bioerodible material comprises carboxymethyl cellulose, trehalose, polyvinylpyrrolidone, maltodextrin, silk, hyaluronic acid, poly(lactic-co-glycolic acid), poly(lactic acid), poly(vinyl alcohol), polyethylene glycol, or a combination of any two or more of the preceding.

According to a thirty-ninth embodiment or aspect, provided is the method of the thirty-fifth or thirty-sixth embodiment or aspect, wherein at least a portion of the stem is formed from a non-dissolvable material.

According to a fortieth embodiment or aspect, provided is the method of any one of the thirty-fifth through the thirty-ninth embodiment or aspect, further comprising delivering a second dissolvable material into the production mold to form a dissolving layer at a portion of the stem, such as adjacent to the microneedle head.

According to a forty-first embodiment or aspect, provided is the method of the fortieth embodiment or aspect, wherein the second dissolvable material of the dissolving layer is a material that dissolves more quickly than first dissolvable or bioerodible material comprising trehalose in water.

According to a forty-second embodiment or aspect, provided is the method of the forty-first embodiment or aspect, wherein the second dissolvable material comprises a low molecular weight compound or composition that rapidly dissolves, e.g., dissolves in less than 30 seconds, less than 20 seconds, less than 10 seconds, or less than 5 seconds in water.

According to a forty-third embodiment or aspect, provided is the method of the forty-first embodiment or aspect, wherein the second dissolvable material comprises glucose, trehalose, sucrose, maltodextrin, polyvinylpyrrolidone, or a combination of two or more of the preceding.

According to a forty-fourth embodiment or aspect, provided is the method of any one of the thirty-fifth through the forty-third embodiment or aspect, wherein the Adenovirus is an Ad5 vector for expression of a transgene.

### BRIEF DESCRIPTION OF THE DRAWINGS

FIGS. 1A and 1B depict schematically exemplary microneedle structures.

FIGS. 2A-2B: Computer-aided design (CAD) drawing of MNAs that include sharp microneedles with undercut features. Geometric parameters of unique microneedle design are depicted on a 2D-CAD drawing (FIG. 2A). 3D-CAD drawing of a 5×5 MNA (FIG. 2B).

FIG. 3: Microneedle array manufacturing strategy involves six distinct steps. (1) 3D-CAD drawing of the target MNA design. (2) Direct production of the master MNA from the 3D-CAD drawing by 3D direct laser printing. (3) High-fidelity replication of the master MNA using a two-step micromolding approach. (4) Creation of the MNA master molds consisting of multiple master MNA replicas (e.g., six MNA replicas) on 3D-printed MNA holders. (5) Manufacturing of the MNA production molds from PDMS using micromolding. (6) Fabrication of tip-loaded, dissolving undercut MNAs incorporating the target vaccine using the spin-casting method.

FIG. 4: Steps of the tip-loading process used for MNA fabrication. (FIG. 4 (A)) Biocargo solution is dispensed onto microneedles in PDMS production molds. (FIG. 4 (B)) Microneedle wells of MNA molds are filled with biocargo by brief centrifugation without drying (bucket lids on). (FIG. 4 (C)) Excess biocargo solution was recovered from the production mold reservoir to be used for fabricating additional MNAs. (FIG. 4 (D)) Remaining biocargo in microneedle molds is dried through spin-casting.

FIG. 5: Summary of fabrication approaches. (A) Traditional engineering approach for the production of undercut structures involves multiple processing and precision assembly steps with the assumption that undercut structures cannot be removed from the molds. (B) Our study demonstrates that it is possible to remove the dissolving undercut microneedles loaded with biocargo from flexible production molds in a single step, resulting in a simpler approach for fabrication of dissolvable undercut MNAs.

FIG. 6: Fabrication of novel dissolving MNAs with undercut microneedles. Final products corresponding to each step of the presented manufacturing strategy. Scale bar is 10 mm (FIG. 6 (A)). Geometric quality control of the fabricated MNAs using optical stereomicroscopy (FIG. 6 (B-I)). Scale bars are 250 μm. Master MNA created using 3D direct laser writing (FIG. 6 (B)). Replica of the master MNA created through a two-stage micromolding strategy (elastomer molding combined with UV-curable micromolding) (FIG. 6 (C)). Microneedle-shaped wells in an MNA production mold (FIG. 6 (D)). Final dissolving CMC/trehalose MNA incorporating a multicomponent vaccine (OVA+Poly(I:C)) (FIG. 6 (E)). Higher magnification of an individual undercut microneedle on the 3D printed master MNA (as in FIG. 6 (B)) (FIG. 6 (F)). Higher magnification of an individual undercut microneedle on master MNA replica (as in FIG. 6 (C)) (FIG. 6 (G)). Final dissolving PVP/PVA undercut microneedle tip-loaded with Alexa680-labeled OVA (FIG. 6 (H)). Final dissolving CMC/trehalose undercut microneedle tip-loaded with doxorubicin, a chemotherapeutic small molecule drug (FIG. 6 (I)).

FIG. 7: Successful fabrication of dissolving MNAs with undercut microneedles integrating single or multiple cargos from different dissolvable biomaterial compositions. Scale bars are 250 μm. PVP/PVA MNAs incorporating Texas Red-labeled dextran (40 kDa MW) at the tips of microneedles (FIG. 7 (A)). Tip-loaded CMC/trehalose MNAs integrating Allura Red R40 dye (˜500 Da MW) at the pyramid region of microneedles (FIG. 7 (B)). Tip-loaded PVP/PVA MNAs incorporating multiple cargos, such as Texas Red-labeled dextran and Allura Red R40 dye (FIG. 7 (C)). Tip-loaded PVP/PVA MNAs incorporating multiple cargos, such as Texas Red-labeled dextran and green fluorescent PLGA microspheres (FIG. 7 (D)).

FIG. 8: Geometric capability of 3D direct laser writing for fabricating microneedles. The additive manufacturing approach that enabled fabrication of novel dissolving MNAs introduced by this study is capable of manufacturing high-quality microneedles with diverse geometries. Different microneedle designs fabricated from nondissolvable UV curable resin (IP-S photoresist; same as for master MNAs) using the Nanoscribe (FIG. 8 (A)). Scale bar is 1 mm. Close-up image of printed microneedles with filleted bases (first five from FIG. 8 (A)) (FIG. 8 (B)). Scale bar is 500 μm.

FIG. 9 (original images in color): Intradermal vaccine delivery to freshly-excised human skin explants using tip-loaded, dissolving MNAs with undercut microneedles. Optical stereomicroscopy images of PVP/PVA MNAs incorporating Allura Red R40 dye before (FIG. 9 (A)) and after (FIG. 9 (B)) application to human skin explants. Scale bars are 250 μm. Optical stereomicroscopy image of Allura Red R40 dye microneedle traces on living human skin samples (FIG. 9 (C)). Scale bar is 500 μm. (FIG. 9 (D-I)) Intradermal co-delivery of Alexa488-labeled Poly(I:C) and Alexa555-labeled OVA from tip-loaded CMC/trehalose MNAs. Scale bars are 100 μm. Fluorescence microscope composite images demonstrate delivery cavities penetrating the epidermis and upper dermis, and delivery of both antigen and adjuvant to targeted skin microenvironments. (FIG. 9 (D)) DAPI nuclear stain. (FIG. 9 (E)) Alexa488-labeled Poly(I:C). (FIG. 9 (F)) Alexa555-labeled OVA. (FIG. 9 (G)) Brightfield. (FIG. 9 (H)) Merged fluorescent images from (FIG. 9 (D-F)). (FIG. 9 (I)) Merged fluorescent images from (FIG. 9 (D-F)) overlaid on brightfield image from (FIG. 9 (G)). (FIG. 9 (J)) Low magnification merged fluorescent images showing OVA and Poly(I:C) delivered in two parallel microneedle tracks. Scale bar is 200 μm.

FIG. 10 (original images in color): MNA directed intradermal vaccine delivery in mice. Delivery kinetics for OVA and Poly(I:C) in murine skin (FIG. 10 (A)). OVA+Poly(I:C) MNAs were applied for 5, 10, or 20 min, and then delivery efficiency of OVA and Poly(I:C) with respect to time was quantified. Data represent percent of initial MNA content delivered (mean±SD, N=6). One-way ANOVA and Tukey's post-hoc tests were used for each biocargo, and significant differences are indicated by *** p<0.001. Optical stereomicroscopy image of MNAs integrating both Alexa555-OVA (red) and Alexa488-Poly(I:C) (green) (FIG. 10 (B)). Scale bar is 500 μm. The fluorescence microscopy inset shows the distribution of both vaccine components in a pyramid microneedle tip. Representative optical stereomicroscopy image of the Alexa555-OVA and Alexa488-Poly(I:C)-loaded MNAs after application to murine skin (FIG. 10 (C)). Scale bar is 250 μm. (FIG. 10 (D-E)) Effective co-delivery of Alexa488-Poly(I:C) (FIG. 10 (D)) and Alexa555-OVA (FIG. 10 (E)) to the mouse skin using MNAs with undercut features.

FIGS. 11A-11C: Intradermal delivery of antigen (OVA)±adjuvant (Poly(I:C)) with MNAs induces antigen-specific cellular and humoral immunity. Mice were immunized by intramuscular (IM) injection of OVA (2×10 μg injections per mouse), or by application of OVA±Poly(I:C) MNAs (10 μg OVA±25 μg Poly(I:C) per MNA, 2 MNAs per mouse) to abdominal skin, and boosted identically seven days later. To determine activity of OVA-specific cytotoxic T lymphocytes (CTLs), equal numbers of unpulsed splenocytes (CFSE^{low} “control” cells) and OVA257-264 peptide-pulsed splenocytes (CFSE^{high} “target” cells) were transferred to naïve and immunized mice (2×10⁷ total cells per mouse) five days later. Spleens and serum were isolated the next day. Representative flow cytometry histograms showing remaining CFSE-labeled cells in spleens of immunized and unimmunized mice. Specific lysis of peptide-pulsed target cells by OVA-specific CTLs is indicated by a reduction in CFSE^{high} target cells (FIG. 11A). Quantification of specific cell lysis, with 100% lysis corresponding to complete elimination of target cells (mean±SD, N=3 mice per group) (FIG. 11B). Serum concentrations of OVA-specific IgG1 and IgG2c antibodies (bars represent mean values, 3 mice per group) (FIG. 11C). Groups were compared by one-way ANOVA, followed by Tukey's post-hoc tests (FIG. 11B), or Dunnett's comparisons to OVA IM control group (FIG. 11C). Significant differences are indicated by * p<0.05, ** p<0.01, or *** p<0.001.

FIGS. 12A-12F. MNAs effectively penetrate the skin and deliver live adenovector vaccines and Poly(I:C) to the same cutaneous microenvironment, driving robust antigen transgene expression. Dissolvable MNAs incorporating Ad.OVA±Poly(I:C) were fabricated using a spincasting method, applied to mouse skin for 10 min, and then removed. Images of MNAs (FIG. 12A) before and (FIG. 12B) after application were obtained using optical stereomicroscopy. Scale bars=500 μm. In vivo multicomponent vaccine delivery performance of MNAs was evaluated by fluorescent live animal imaging following application of MNAs incorporating Alexa488-labeled Poly(I:C) and Alexa555-labeled Ad.OVA to the right ears of mice. Mice were imaged using the IVIS 200 system with filters corresponding to (FIG. 12C) Alexa488 and (FIG. 12D) Alexa555 to demonstrate simultaneous codelivery of Ad.OVA and Poly (I:C). (FIG. 12E) MNA-treated mouse skin was excised and imaged by epifluorescent microscopy and brightfield microscopy to show intercutaneous delivery of multicomponent vaccines in vivo. Scale bars=100 μm. (FIG. 12F) To quantify transgene (OVA) expression in the skin, mouse skin treated with Ad.OVA±Poly(I:C) MNAs was recovered after 24, 48, and 72 hr, and OVA mRNA expression in the skin was quantified by qRT-PCR. Data are presented as mean±standard deviation. Significance was determined by two-way ANOVA followed by Sidak multiple comparison test. **p<0.01 and ****p<0.0001.

FIGS. 13A-13G. Intercutaneous immunization with multicomponent MNA vaccine platforms incorporating adenovector-encoded OVA and Poly(I:C) adjuvant more effectively engineers a pro-inflammatory skin microenvironment in vivo, promoting robust immune responses compared to immunization with MNA adenovector vaccine alone. Mice were immunized with Ad.OVA±Poly(I:C) MNAs or blank MNAs (control). Antigen-specific cell-mediated and humoral immune responses were determined at the indicated time points using established lytic and ELISA assays, respectively. To assess stability of multicomponent MNAs, intercutaneous immunization experiments were repeated with Ad.OVA+Poly(I:C) MNAs stored at 4° C. for one month. (FIG. 13A) Quantification of OVA-specific lytic responses. (FIGS. 13B and 13C) Quantification of serum concentrations of OVA-specific IgG1 and IgG2c antibodies, respectively. Data are presented as mean±standard deviation and analyzed by one-way ANOVA, followed by Tukey's post-hoc test. ns>0.05, * p<0.05, ** p<0.01, **** p<0.0001. (FIGS. 13D-13G) To investigate key immune mediators in the skin microenvironment induced by immunization, MNAs with the indicated components or blank MNAs were applied as described above, and expression of (FIG. 13D) IFNB1, (FIG. 13E) CXCL10, (FIG. 13F) IL-1p and (FIG. 13G) IL-6 mRNA levels was quantified by qRT-PCR at the indicated time points. Data are presented as mean±standard deviation and analyzed by two-way ANOVA, followed by Tukey's multiple comparisons test. Significant differences between treatment groups at each timepoint are indicated by * p<0.05, ** p<0.01, *** p<0.001. FIG. 13A provides a color key for FIGS. 13A-31G, with, from lightest to darkest, for FIGS. 13A-13C, from left to right: Blank; Ad.OVA; Ad.OVA+Poly(I:C); and Ad.OVA+Poly(I:C) after one-month storage, and for FIGS. 13D-13G, each grouping of columns per time point, from left to right, representing: Blank; Poly (I:C); Ad.OVA; Ad.OVA+Poly(I:C); and Ad.OVA+Poly(I:C) after one-month storage.

### DESCRIPTION OF THE INVENTION

Provided herein are microneedle devices, e.g., microneedle arrays (MNAs), for delivery of recombinant adenovirus particles to the skin of a patient. The microneedle devices retain significant activity, e.g., retain substantial numbers of infectious adenovirus particles (e.g., infectous units, or IUs), and/or retain substantial therapeutic effect, on storage at above-freezing temperatures, enabling long-term storage and less-stringent distribution purposes, such as 4° C. for at least one month when hermetically-sealed. A further benefit is the ease of use of the microneedle devices, obviating the need for syringes and needles. Lastly, for immunogen delivery, as in the case of a vaccine, intradermal delivery may be preferred. In aspects, the microneedle device described herein comprises microneedles comprising adenovirus particles, trehalose, optionally an adjuvant, and typically another biodegradable or bioerodable compound or composition such as carboxymethylcellulose and/or a hydrogel polymer.

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. Further, as used herein, all numbers expressing dimensions, physical characteristics, processing parameters, quantities of ingredients, reaction conditions, and the like, used in the specification and claims are to be understood as being modified in all instances by the term “about”. Moreover, unless otherwise specified, all ranges disclosed herein are to be understood to encompass the beginning and ending range values and any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 3.3, 4.7 to 7.5, 5.5 to 10, and the like.

As used herein “a” and “an” refer to one or more. The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention.

As used herein, spatial or directional terms, such as “left”, “right”, “inner”, “outer”, “above”, “below”, “over”, “under”, and the like, relate to the invention as it is shown in the drawing figures are provided solely for ease of description and illustration, and do not imply directionality, unless specifically required for operation of the described aspect of the invention. It is to be understood that the invention can assume various alternative orientations and, accordingly, such terms are not to be considered as limiting.

As used herein, a “patient” or “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). As used herein, the terms “treating”, or “treatment” refer to a beneficial or desired result, such as improving one of more functions, or symptoms of a disease.

Unless otherwise explained, all technical and scientific terms used herein have the same meaning as commonly understood by one of ordinary skill in the art to which this disclosure belongs. It is to be understood that all base sizes or amino acid sizes, and all molecular weight or molecular mass values, given for nucleic acids or polypeptides are approximate, and are provided for description. Unless otherwise indicated, polymer molecular weight is expressed as number-average molecular weight (Mn). Although methods and materials similar or equivalent to those described herein can be used in the practice or testing of the present disclosure, suitable methods and materials are described below.

Unless stated otherwise, nucleotide sequences are recited herein in a 5′ to 3′ direction, and amino acid sequences are recited herein in an N-terminal to C-terminal direction according to convention.

“Packaging” refers to any container in which an object can be stored and/or distributed, including, for example and without limitation: vessels, boxes, medical syringes, pouches, tubes, flasks, bottles, and/or wrappings. Packaging may be hermetically sealed, or airtight, such that water vapor and air cannot pass into or from the inside of the packaging. For example, a microneedle device may be hermetically-sealed within a Mylar pouch, optionally with an inert atmosphere within the pouch such as nitrogen or argon, or vacuum-sealed, thereby preventing the microneedle device from losing or gaining water content and/or oxidation of constituents of the device. A device as described herein may be stored and transferred between locations in packaging and at a suitable temperature for retaining activity of the therapeutic agent, e.g. the adenovirus particles, in the device. For example, the device may be stored and distributed under refrigeration at 4° C. The packaging may be a hermetically-sealed package (e.g., Mylar pouch), optionally with an inert (e.g., N₂ or Ar, or vacuum) atmosphere.

“Therapeutically effective amount,” as used herein, is intended to include the amount of a therapeutic agent, such as an immunogen, as described herein that, when administered to a subject having a disease, is sufficient to effect treatment of the disease (e.g., by diminishing, ameliorating or maintaining the existing disease or one or more symptoms of disease). Alternatively, the effective amount may be an amount effective to deliver an amount of an immunogen effective to elicit a desired immune response, e.g., to elicit in a single treatment, or in multiple treatments (e.g., prime-boost), protective immunity against a pathogen, such as a virus. The “therapeutically effective amount” may vary depending on the compound or composition, how it is administered, the disease and its severity and the history, age, weight, family history, genetic makeup, the types of preceding or concomitant treatments, if any, and other individual characteristics of the subject to be treated. A therapeutically-effective amount need not produce 100% efficacy in a population of patients.

A “therapeutically-effective amount” also includes an amount of an agent that produces some desired local or systemic effect at a reasonable benefit/risk ratio applicable to any treatment. Compounds and compositions described herein may be administered in a sufficient amount to produce a reasonable benefit/risk ratio applicable to such treatment. For example, a therapeutically-effective amount of a vaccine useful for eliciting an immune response in a subject and/or for preventing infection by a pathogen in a patient, or in a statistically significant number of patients. A therapeutically effective amount of a vaccine is an amount sufficient to increase resistance to, prevent, ameliorate, and/or treat infection caused by a pathogen. Because the described virus particles comprise a gene for expression of a therapeutic polypeptide, such as an immunogen, the gene may be subject to transcriptional control of a variety of promoters or transcription response elements (TREs) of varying potency. As such, the number of adenoviral particles loaded into each microneedle may vary greatly, ranging from 1 to 10¹⁴ viral particles (e.g., infections units (IU)) per microneedle, such as 1, 10, 10², 10³, 10⁴, 10⁵, 10⁶, 10⁷, 10⁸, 10⁹, 10¹⁰, 10¹¹, 10¹², 10¹³, or 10¹⁴ viral particles or IUs per microneedle.

Dosage can be varied to maintain a desired concentration at a target site (for example, locally). Higher or lower concentrations can be selected based on the mode of delivery, for example, trans-epidermal, rectal, oral, pulmonary, or intranasal delivery versus intravenous or subcutaneous delivery. The actual dosage of disclosed adenoviral particles may vary according to factors such as the disease indication and particular status of the subject (for example, the subject's age, size, fitness, extent of symptoms, susceptibility factors, and the like), dosage regimen, other drugs or treatments being administered concurrently, as well as the specific pharmacology of the delivered polypeptide for eliciting the desired activity or biological response in the subject. Dosage regimens can be adjusted to provide an optimum prophylactic or therapeutic response. A therapeutically effective amount is also one in which any toxic or detrimental side effects of the therapeutic polypeptide or immunogen and/or other biologically active agent is outweighed in clinical terms by therapeutically beneficial effects.

Further, preventing, treating or ameliorating a disease: “Preventing” a disease refers to inhibiting the full development of a disease or infection from a subsequent exposure. “Treating” refers to a therapeutic intervention that ameliorates a sign or symptom of a disease or pathological condition after it has begun to develop. “Ameliorating” refers to the reduction in the number or severity of one or more signs or symptoms of a disease or infection. Prime-boost vaccination: an immunotherapy including administration of a first immunogenic composition (the primer vaccine) followed by administration of a second immunogenic composition (the booster vaccine) to a subject to elicit an immune response. The primer vaccine and/or the booster vaccine include, in the context of the present disclosure, an adenoviral particle expressing an antigen to which the immune response is directed. The booster vaccine is administered to the subject after the primer vaccine; the skilled artisan will understand a suitable time interval between administration of the primer vaccine and the booster vaccine. The primer vaccine, the booster vaccine, or both primer vaccine and the booster vaccine additionally may include an adjuvant.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (24) other non-toxic compatible substances employed in pharmaceutical formulations.

As used herein, administering a composition (e.g., an immunogenic composition, such as a vaccine) to a subject means to give, apply or bring the composition into contact with the subject. Administration can be accomplished by any of a number of routes, such as, for example, topical, oral, subcutaneous, intradermal intramuscular, intraperitoneal, intravenous, intrathecal, and intramuscular. In the context of the present disclosure, relating to microneedle devices, the delivery route is predominantly to the skin, and may be, for example and without limitation, intradermal, cutaneous, transdermal, or subcutaneous, depending on the size of the needles and thickness of the patient's skin. Delivery of a therapeutic polypeptide to a patient's skin can provide sufficient amounts of a therapeutic polypeptide, for example an immunogen, to a patient for local, and even systemic therapies.

A biological sample is a sample obtained from a subject (such as a human or veterinary subject). Biological samples, include, for example, fluid, cell and/or tissue samples. In some embodiments herein, the biological sample is a fluid sample. Fluid sample include, but are not limited to, serum, blood, plasma, urine, feces, saliva, cerebral spinal fluid (CSF), and bronchoalveolar lavage (BAL) fluid.

The term “contacting” refers to placement in direct physical association; includes both in solid and liquid form. “Contacting” is often used interchangeably with “exposed”, or, depending on context, “administering.” In some cases, “contacting” includes transfecting, such as transfecting a nucleic acid molecule into a cell. In other examples, “contacting” refers to incubating a molecule (such as an antibody) with a biological sample.

A fusion protein or fusion polypeptide refers to a protein or polypeptide generated, for example, by expression of a nucleic acid sequence engineered from nucleic acid sequences encoding at least a portion of two different (heterologous) proteins. To create a fusion protein, the nucleic acid sequences are in the same reading frame and contain no internal stop codons.

An immune response is a response of a cell of the immune system, such as a B-cell, T-cell, macrophage or polymorphonucleocyte, to a stimulus, such as an antigen. An immune response may include any cell of the body involved in a host defense response for example, an epithelial cell that secretes an interferon or a cytokine. An immune response includes, but is not limited to, an innate immune response or inflammation.

An immunogen refers to a compound, composition, or substance which is capable, under appropriate conditions, of stimulating an immune response, such as the production of antibodies, such as neutralizing antibodies, and/or a T-cell response in an animal, including compositions that are injected or absorbed into an animal. As used herein, an “immunogenic composition” is a composition comprising an immunogen.

An “isolated” or “purified” biological component (such as a nucleic acid, peptide, protein, protein complex, or particle) refers to a component that has been substantially separated, produced apart from, or purified away from other components in a preparation or other biological components in the cell of the organism in which the component occurs, that is, other chromosomal and extrachromosomal DNA and RNA, and proteins. Nucleic acids, peptides and proteins that have been “isolated” or “purified” thus include nucleic acids and proteins purified by standard purification methods. The term also embraces nucleic acids, peptides and proteins prepared by recombinant expression in a host cell, as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell, or other production vessel. A preparation may be purified such that the biological component represents at least 50%, such as at least 70%, at least 90%, at least 95%, or greater, of the total biological component content of the preparation.

A nucleic acid molecule (a nucleic acid) refers to a polymeric form of nucleotides, which may include sense and/or anti-sense strands of RNA, cDNA, genomic DNA, and synthetic forms and mixed polymers of the above. A nucleotide refers to a ribonucleotide, a deoxyribonucleotide, or a modified form of either type of nucleotide. The term “nucleic acid molecule” as used herein is synonymous with “nucleic acid” and “polynucleotide.” The term includes single- and double-stranded forms of DNA. A polynucleotide may include either or both naturally occurring and modified nucleotides linked together by naturally occurring and/or non-naturally occurring nucleotide linkages.

A first nucleic acid is said to be operably linked to a second nucleic acid when the first nucleic acid is placed in a functional relationship with the second nucleic acid. Generally, operably linked DNA sequences are contiguous (e.g., in cis) and, where the sequences act to join two protein coding regions, in the same reading frame. Operably linked nucleic acids include a first nucleic acid contiguous with the 5′ or 3′ end of a second nucleic acid. In other examples, a second nucleic acid is operably linked to a first nucleic acid when it is embedded within the first nucleic acid, for example, where the nucleic acid construct includes (in order) a portion of the first nucleic acid, the second nucleic acid, and the remainder of the first nucleic acid.

A polypeptide is a polymer in which the monomers are amino acid residues which are joined together through amide bonds. When the amino acids are alpha-amino acids, unless specified, either the L-optical isomer or the D-optical isomer can be used. The terms “polypeptide” or “protein” as used herein are intended to encompass any amino acid sequence and include proteins and modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

A promoter is an array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor).

A recombinant nucleic acid refers to a nucleic acid molecule (or protein or virus) that is not naturally occurring or has a sequence that is made by an artificial combination of two otherwise separated segments of sequence. This artificial combination is accomplished by chemical synthesis or, more commonly, by the artificial manipulation of isolated segments of nucleic acids, e.g., by genetic engineering techniques as are broadly-known. The term recombinant includes nucleic acids and proteins that have been altered solely by addition, substitution, or deletion of a portion of a natural nucleic acid molecule or protein.

A “transformed” cell is a cell into which has been introduced a nucleic acid molecule (such as a heterologous nucleic acid) by any useful molecular biology technique. The term encompasses all techniques by which a nucleic acid molecule might be introduced into such a cell, including, without limitation, transfection with viral vectors (e.g., adenoviral vectors), transformation with plasmid vectors, and introduction of naked DNA by electroporation, lipofection, or particle gun acceleration.

A vaccine refers to a preparation of immunogenic material capable of stimulating an immune response, administered for the prevention, inhibition, amelioration, or treatment of infectious, or other types of disease. The immunogenic material may include attenuated or inactivated (killed) microorganisms (such as bacteria or viruses), or antigenic proteins, peptides, or DNA derived from them. Vaccines may elicit both prophylactic (preventative or protective) and therapeutic responses. Methods of administration vary according to the vaccine, but may include inoculation, ingestion, inhalation or other forms of administration, though in the context of the present disclosure, by delivery via the described microneedle devices. Vaccines may be administered with an adjuvant to boost the immune response.

A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication, or may omit or modify such sequences, such that the vector cannot replicate or only can go through a limited, e.g., single, round of replication (e.g., replication defective). An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes.

By “expression” or “gene expression,” it is meant the overall flow (processing) of information from a gene to produce a gene product (typically a protein, optionally post-translationally modified, or a functional/structural RNA). A “gene” refers to a functional genetic unit for producing a gene product, such as RNA or a protein, in a cell or other expression system. A gene is encoded on a nucleic acid and comprises: a transcriptional control sequence, such as a promoter and other cis-acting elements, such as transcriptional response elements (TREs) and/or enhancers; an expressed sequence that may encode a protein (referred to as an open-reading frame or ORF) or a functional RNA, and a polyadenylation sequence. By “expression of genes under transcriptional control of,” or alternately “subject to control by,” a designated sequence such as TRE or transcription control element, it is meant gene expression from a gene containing the designated sequence operably linked (functionally attached, typically in cis) to the gene. A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a suitable host cell. In the case of an inducible promoter, “suitable conditions” means when factors that regulate transcription, such as DNA-binding proteins, are present or absent in the cell or expression system—for example an amount of the respective inducer is available to the expression system (e.g., cell), or factors causing suppression of a gene are unavailable or displaced—effective to cause expression of the gene. A “transgene” is a gene from one organism expressed in a cell of another organism.

In the devices and methods described herein, an adenoviral vectors (adenovirus particles) that express a therapeutic polypeptide, such as an immunogen, is incorporated into the microneedle device. The adenoviral DNA comprises a gene encoding the polypeptide to be expressed, such as an immunogen. One of skill in the art can readily use the genetic code to construct a variety of functionally equivalent nucleic acids, such as nucleic acids which differ in sequence but which encode the same protein sequence, or encode a conjugate or fusion protein including the nucleic acid sequence. In some embodiments, the polynucleotide is codon-optimized for expression in human cells. In specific non-limiting examples, adenoviral vectors comprising nucleic acids comprising a gene encoding a coronavirus, e.g., SARS-CoV-2, immunogen, such as a spike protein, a portion thereof, or a polypeptide comprising an epitope thereof, may be provided.

Exemplary nucleic acids may be prepared by cloning techniques, as are broadly-known and implemented either commercially, or in the art. Multiple textbooks and reference manuals describe and provide examples of useful and appropriate cloning and sequencing techniques, and instructions sufficient to direct persons of skill through such techniques are known. Commercial and public product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (Carlsbad, Calif.), Addgene, and Applied Biosystems (Foster City, Calif.), as well as many other commercial sources.

The terms “iRNA,” “RNAi reagent,” and “RNA interference agent” as used interchangeably herein, refer to an agent that contains RNA nucleotides, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., knocks down or silences, the expression of RNA in a cell, e.g., a cell within a subject, such as a mammalian subject.

Two types of short RNA molecules have been used in RNAi applications. Small interfering RNA (siRNA) are typically double-stranded RNA molecules, 20-25 nucleotides in length. When transfected into cells, siRNA inhibit the target mRNA transiently until they are also degraded within the cell. Small hairpin RNAs (shRNA) are sequences of RNA, typically about 80 base pairs in length, that include a region of internal hybridization that creates a hairpin structure. shRNA molecules are processed within the cell to form siRNA which in turn knock down gene expression. The benefit of shRNA is that they can be incorporated into plasmid vectors, such as recombinant adenovirus vectors and particles as described herein. An example of an adenoviral vector system for expression of shRNAs is The pAd/BLOCK-iT™-DEST RNAi Gateway® Vector, commercially available from ThermoFisher Scientific (see, also, for example, Pei, Z., et al. Adenovirus vectors lacking virus-associated RNA expression enhance shRNA activity to suppress hepatitis C virus replication. Sci Rep 3, 3575 (2013)). As with shRNA, antisense sequences may be expressed by the gene of the recombinant adenovirus particles described herein. Furthermore, gene editing nucleic acids and/or proteins, such as CRISPR/Cas9 machinery, may be encoded by one or more genes of the adenoviral particles (see, e.g., Ehrke-Schulz, E., et al. CRISPR/Cas9 delivery with one single adenoviral vector devoid of all viral genes. Sci Rep 7, 17113 (2017)).

The disclosed viral vectors may comprise a gene for expressing a therapeutic polypeptide, e.g., an immunogen, can be delivered to a subject. In the case of an immunogen, to produce an immune response to a pathogen or cancer cell. The pathogen may be a coronavirus, such as a SARS-CoV-2 virus, for the purpose of eliciting an immune response, such as a protective or neutralizing immune response.

Delivery may be transcutaneous via the microneedle arrays (MNAs) microneedle devices described herein. Adenoviral-based vaccines against human immunodeficiency virus (HIV), Ebola virus, influenza virus, Mycobacterium tuberculosis, and Plasmodium falciparum are currently under review, and are suited for delivery by the microneedle devices described herein. Additional adenoviral vector vaccines incorporating antigens obtained from emerging or existing pathogenic threats may be developed and are equally suited for delivery via the devices and methods described herein.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In the adenoviral vectors described herein, polynucleotide sequences encoding a disclosed therapeutic polypeptide, such as an immunogen, can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, TREs, enhancers, transcription terminators, a start codon (e.g., ATG) in front of a protein-encoding gene, splicing signal for introns, maintenance of the correct reading frame of that gene to permit proper translation of mRNA, and stop codons.

In preparation and propagation of nucleic acid sequences encoding the disclosed polypeptides, those nucleic acid sequence may be expressed in vitro by DNA transfer into a suitable host cell. The cell may be prokaryotic or eukaryotic. Host cell also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known. Examples of commonly used mammalian host cell lines are VERO and HeLa cells, CHO cells, and W138, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC No. CRL-3022), or HEK-293F cells.

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂) method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. One of skill in the art can readily use an expression systems such as plasmids and vectors of use in producing proteins in cells including higher eukaryotic cells such as the COS, CHO, HeLa and myeloma cell lines.

Modifications may be made to a nucleic acid encoding a polypeptide without diminishing its biological activity. Some modifications may be made to facilitate the cloning, expression, or incorporation of the targeting molecule into a fusion protein. Such modifications include, for example, termination codons, a methionine added at the amino terminus to provide an initiation site, additional amino acids placed on either terminus to create conveniently located restriction sites, or additional amino acids (such as poly-His) to aid in purification steps.

A nucleic acid molecule encoding a disclosed immunogen may be included in a viral vector, for example, for expression of the immunogen in a host cell, or for immunization of a subject as disclosed herein. In some embodiments, the viral vectors are administered to a subject as part of a prime-boost vaccination. The viral vectors may be included in a vaccine, such as a primer vaccine or a booster vaccine for use in a prime-boost vaccination.

The viral vector may be replication-competent, conditionally replication-competent or replication-deficient in host cells or in therapeutic target cells. In the context of the present disclosure, the viral vector is an adenoviral vector that expresses a therapeutic polypeptide, such as an immunogen. Methods of making, propagating, and using adenovirus vectors and adenovirus particles are broadly-known, with many suitable vectors being described in publications and being available commercially. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) may be used to generate the adenoviral vector. For example, a simian adenovirus can be used as the source of the viral genome of the adenoviral vector. A simian adenovirus may be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. A chimpanzee serotype C Ad3 vector may be used (see, e.g., Peruzzi et al., Vaccine, 27:1293-1300, 2009) or an Ad5 vector may be used (see the Examples Section). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. The person of ordinary skill in the art is familiar with replication competent and deficient adenoviral vectors (including singly and multiply replication deficient adenoviral vectors). Non-limiting examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/022311.

Non-limiting examples of adenovirus particles that may be delivered by the devices and methods described herein include SARS-CoV-2 adenoviral vaccines, including, for example and without limitation: ChAdOx1 nCoV-19 (AstraZeneca), and variations thereof, e.g., to account for viral mutations, and Ad26.COV2.S (Johnson & Johnson/Janssen), and variations thereof, e.g., to account for viral mutations.

The devices and methods disclosed herein, e.g., when used for delivery of an immunogen, may include one or more compound or composition having immune stimulant or adjuvant effect. In other examples, a compound or composition having immune stimulant or adjuvant effect is not included in the composition, but is separately administered to a subject (for example, in combination with a composition disclosed herein) before, after, or substantially simultaneously with administration of one or more of an immunogen-containing composition as disclosed herein. A compound or composition having immune stimulant or adjuvant effect is a compound or composition that increases or enhances an immune response in a subject administered an antigen, compared to administration of the antigen in the absence of a compound or composition having immune stimulant or adjuvant effect. A compound or composition having immune stimulant or adjuvant effect may be a compound or composition referred to as an adjuvant. One example of a compound or composition having immune stimulant or adjuvant effect is an aluminum salt, such as aluminum hydroxide, aluminum phosphate, aluminum potassium sulfate, or aluminum hydroxyphosphate. Other compounds or compositions having immune stimulant or adjuvant effect include biological adjuvants, such as cytokines (for example, IL-2, IL-6, IL-12, RANTES, GM-CSF, TNF-α, or IFN-γ), growth factors (for example, GM-CSF or G-CSF), one or more molecules such as OX-40L or 4-1 BBL, immunostimulatory oligonucleotides (for example, CpG oligonucleotides, for example, see U.S. Pat. Nos. 6,194,388; 6,207,646; 6,214,806; 6,218,371; 6,239,116; 6,339,068; 6,406,705; and 6,429,199), Toll-like receptor agonists (for example, TLR2, TLR4, TLR7/8, or TLR9 agonists), and bacterial lipopolysaccharides or their derivatives (such as 3D-MPL). Additional compounds or compositions having immune stimulant or adjuvant effect include oil and water emulsions, squalene, or other agents. A compound or composition having immune stimulant or adjuvant effect may be a water-in-oil emulsion in which antigen solution is emulsified in mineral oil (for example, Freund's incomplete adjuvant), sometimes with the inclusion of killed mycobacteria (Freund's complete adjuvant) to further enhance antigenicity. In one example, a compound or composition having immune stimulant or adjuvant effect is a mixture of stabilizing detergent, micelle-forming agent, and oil available under the name PROVAX® (IDEC Pharmaceuticals, San Diego, Calif.). One of skill in the art can select a suitable compound or composition having immune stimulant or adjuvant effect, or combination of compounds or compositions having immune stimulant or adjuvant effect, to be included in the compositions disclosed herein or administered to a subject in combination with the compositions disclosed herein. Compounds or compositions having immune stimulant or adjuvant effect, include, without limitation: TLR3 agonists such as Poly(I:C) or Poly-ICLC; TLR 4 agonists such as LPS or monophosphoryl lipid derivatives; TLR 5 agonists such as flagellin derivatives; TLR 7/8 agonists such as imiquimod or R848; TLR 9 agonists such as CpG sequences; Stimulator of Interferon Genes (STING) pathway agonists such as ADU-S100; stimulatory neuroimmune mediators such as calcitonin gene-related peptide (CGRP); neurokinin 1 (NK1) receptor agonists such as Hemokinin 1 and Substance P; saponin related adjuvants such as QS-21 (Quillaja saponaria); purinoergic receptor agonists such as ATP; or oil-in-water emulsion adjuvants such as MF59.

Also provided herein are methods of eliciting a therapeutic effect in a patient by administering to the patient an adenoviral particle comprising a gene for expressing the therapeutic polypeptide, as disclosed herein. In a particular example, the subject is a human. Also provided herein are methods of eliciting an immune response in a patient by administering to the subject an adenoviral particle comprising a gene for expressing the immunogen. In a particular example, the subject is a human. The subject can be a human.

In some examples, the method further includes selecting a patient in need of a therapeutic agent, such as enhanced immunity to a pathogen. The patient may be a cancer patient, the patient may be at risk for severe symptoms or death from infection with a pathogen, or the patient may be at high risk for exposure to a pathogen, such as a medical worker, or a person traveling to a region with high risk of contracting malaria.

Dissolvable microneedle arrays enable efficient and safe drug and vaccine delivery to the skin and mucosal surfaces. However, inefficient drug delivery can result from the homogenous nature of conventional microneedle array fabrication. Although the drugs or other cargo that is to be delivered to the patient are generally incorporated into the entire microneedle array matrix, in practice only the microneedles enter the skin and therefore, only cargo contained in the volume of the individual needles is deliverable. Accordingly, the vast majority of the drugs or other cargo that is localized in the non-needle components (e.g., the supporting structure of the array) is never delivered to the patient and is generally discarded as waste.

A fully-dissolvable microneedle array substrate and unique microneedle geometries may be utilized that enable effective delivery of the Adenovirus particles, and therefore the therapeutic agent encoded by the adenoviral vector (genome). This technology can also uniquely enable the simultaneous co-delivery of additional chemically distinct agents for polyfunctional drug delivery, such as different adenovirus particles or compounds or compositions having immune stimulant or adjuvant effect.

A dissolvable microneedle array (microneedle device) for transdermal insertion, e.g., local cutaneous delivery, into a subject may be provided for promoting an immune response against a coronavirus in a subject in need thereof. The array includes a base portion and a plurality of microneedles extending from the base portion and containing a disclosed immunogen, or a vector encoding the immunogen, and optionally at least one adjuvant.

The plurality of microneedles may be pre-formed to have a shape that comprises a first cross-sectional dimension at a top portion, a second cross-sectional dimension at a bottom portion, and a third cross-sectional dimension at an intermediate portion, wherein the intermediate portion is located between the top portion and the bottom portion, and the third cross-sectional dimension is greater than the first and second cross-sectional dimensions. Each microneedle may comprise a plurality of layers of dissoluble biocompatible material, such as, but not limited to carboxymethylcellulose.

A fabrication technology may be utilized that results in various active components to be incorporated into the needle tips, see U.S. Published Patent Application No. US-20160271381 A1, which is incorporated herein by reference. Thus, by localizing the active components in this manner, the remainder of the microneedle array volume includes less expensive matrix material that is non-active, and is preferably generally regarded as safe. The net result is greatly improved efficiency of drug delivery based on (1) reduced waste of non-deliverable active components incorporated into the non-needle portions of the microneedle array, and (2) higher drug concentration in the skin penetrating needle tips.

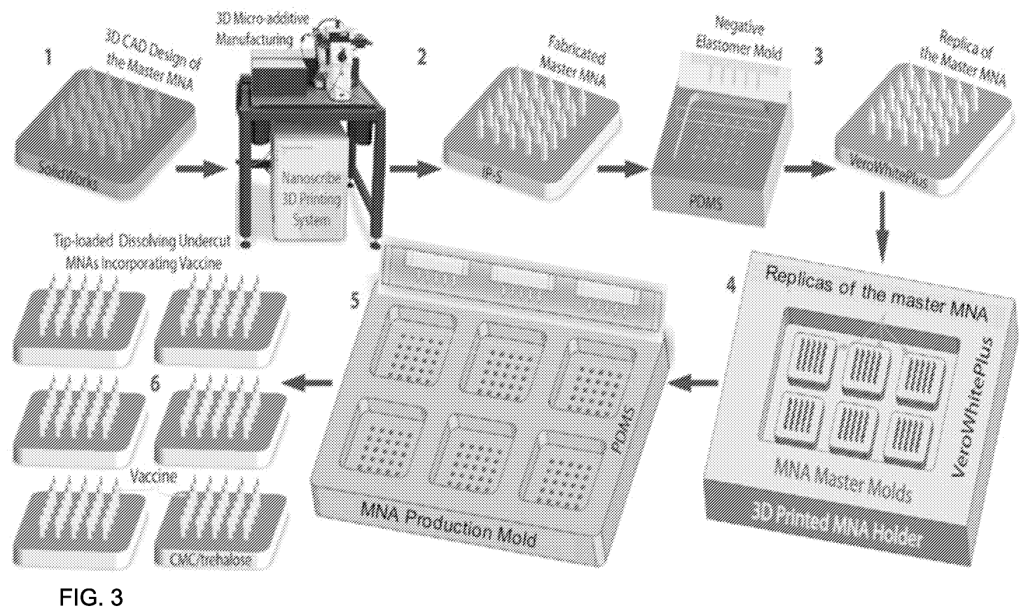

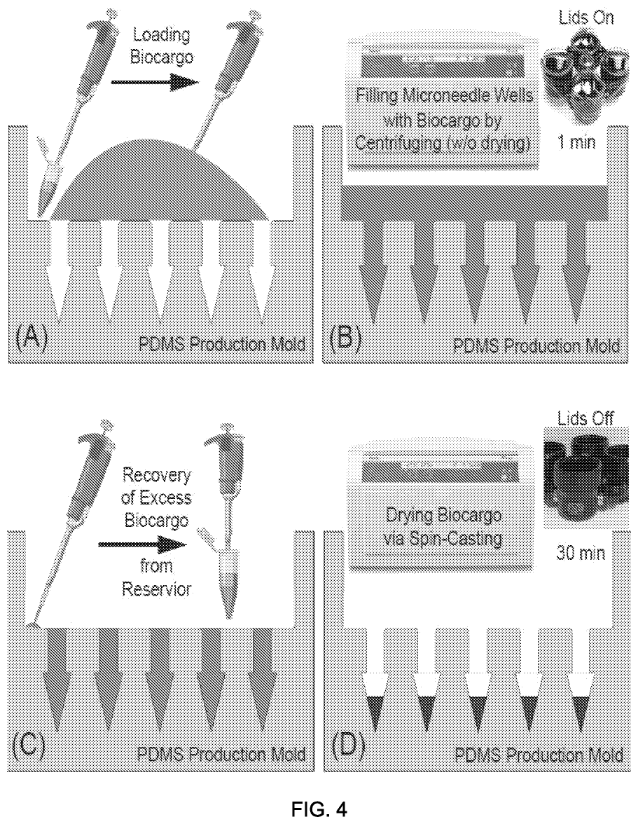

Thus, the active component may be concentrated in the microneedle tips of the respective arrays (microneedle devices). In contrast to conventional microneedle arrays, the active component is not present at even concentration throughout the microneedle array since there is little or no active component present in the supporting base structure. In addition, as shown, for example, in FIGS. 3A, 3B, 4A, and 4B of U.S. Published Patent Application No. US-20160271381 A1, which is incorporated herein by reference, not only is there little or no active component in the supporting structures, the location of the active component is concentrated in the upper half of the individual microneedles in the array. The active component may be concentrated in the upper half of the individual microneedles. The active component may be concentrated in the tip of the microneedle, with the tip being defined by an area of the microneedle that extends from a base portion in a narrowing and/or tapered manner. The base portion, in turn, extends from the supporting structure of the array.

As noted above, individual microneedles may comprise active components only in the upper half (tip) of the microneedle. Individual microneedles may comprise active components only in the tips or in a narrowing portion near the tip of the microneedle. Individual needles may comprise active components throughout the entire microneedle portion that extends from the supporting structure, see U.S. Published Patent Application No. US-20160271381 A1, which is incorporated herein by reference.

The disclosed adenovirus particles also may be delivered essentially as disclosed in PCT Application No. PCT/US2016/057363, which is incorporated herein by reference. That PCT application disclosed microneedle arrays that can be configured to penetrate the stratum corneum to deliver their cargo (e.g., biologics or bioactive components) to the epidermis and/or dermis, while minimizing pain and bleeding by preventing penetration to deeper layers that may contain nerve endings and vessels. Pyramidal CMC-microneedles effectively penetrated the stratum corneum, epidermis, and dermis of living human skin, and, thus, can be used for cutaneous delivery. Thus, the microneedle array may include pyramidal trehalose-containing-microneedles.

To construct the microneedle array devices, a base material may be used to form portions of each microneedle that have bioactive components and portions that do not. As discussed above, each microneedle may comprise bioactive components only in the microneedles, or in some embodiments, only in the upper half of the microneedles, or in other embodiments, only in a portion of the microneedle that tapers near the tip. Thus, to control the delivery of the bioactive component(s) and to control the cost of the microneedle arrays, each microneedle preferably has a portion with a bioactive component (immunogen and/or adjuvant) and a portion without a bioactive component. In the embodiments described herein, the portion without the bioactive component includes the supporting structure of the microneedle array and, in some embodiments, a base portion (e.g., a lower half) of each microneedle in the array.

Various materials may be used as the base material for the microneedle arrays. The structural substrates of biodegradable solid microneedles may include poly(lactic-co-glycolic acid) (PLGA) or carboxymethylcellulose (CMC) based formulations; however, other bases can be used. As disclosed herein, the portion of the microneedles containing the adenovirus particles, and/or a layer adjacent thereto, comprises trehalose. Trehalose may be included in the entire microneedle matrix, or a portion of the microneedle matrix including the adenovirus particles, or a layer adjacent to the portion of the microneedle matrix including the adenovirus particles. The trehalose-containing portions of the matrix may comprise from 0.5% to 100% w/w trehalose, for example, at least 1% w/w, 2% w/w, 5% w/w, 10% w/w, or 15% w/w trehalose, including increments within that range. In one example, the trehalose-containing portions of the matrix comprise from 5% to 20% w/w trehalose, and, optionally from 5% to 25% w/w carboxymethylcellulose. The trehalose-containing portions of the matrix may comprise from 0% to 75% w/w water, for example from 60% to 80% w/w water, e.g., 70% w/w or 75% w/w or approximately 70% w/w or 75% w/w water, with the remainder being trehalose, or a combination of trehalose and a polysaccharide, such as carboxymethylcellulose (e.g., a total solute concentration of 25% w/w or 30% w/w.

CMC may be preferable to PLGA as the base material of the microneedle arrays described herein. The PLGA based devices can limit drug delivery and vaccine applications due to the relatively high temperature (e.g., 135° C., or higher) and vacuum required for fabrication. In contrast, a CMC-based matrix can be formed at room temperature in a simple spin-casting and drying process, making CMC-microneedle arrays more desirable for incorporation of sensitive biologics, peptides, proteins, nucleic acids, and other various bioactive components.

Hydrogel may be prepared from low viscosity sodium salt of CMC with or without trehalose, in sterile dH₂O. CMC can be mixed with trehalose, sterile distilled water (dH₂O) to achieve about 25 to 30 wt % solute concentration. The resulting mixture can be stirred to homogeneity and equilibrated at about 4° C. for 24 hours. During this period, the CMC and any other components may be hydrated and a hydrogel can be formed. The hydrogel may be degassed in a vacuum for about an hour and centrifuged at about 20,000 g for an hour to remove residual micro-sized air bubbles that might interfere with a spincasting/drying process of the CMC-microneedle arrays. The dry matter content of the hydrogel can be tested by drying a fraction (10 g) of it at 85° C. for about 72 hours. The ready-to-use CMC-hydrogel or CMC/trehalose-hydrogel may be stored at about 4° C. until use.

Arrays can be spin-cast at room temperature, making the process compatible with the functional stability of adenovirus particles. Since the master and production molds can be reusable for a large number of fabrication cycles, the fabrication costs can be greatly reduced. The resulting dehydrated CMC/trehalose/adenovirus-microneedle arrays are generally stable at room temperature or slightly lower temperatures (such as about 4° C.), and preserve the activity of the incorporated virus particles, facilitating easy, low cost storage and distribution.

In one example, Adenovirus particles and, optionally a compound or composition having immune stimulant or adjuvant effect may deposited onto the surface of a production mold and spin-casted by centrifugation at 2,500 g for about 5 minutes. The surface of the mold is then covered with about 50 μl (for molds with 11 mm diameter) of CMC/trehalose-hydrogel and spin-casted by centrifugation at 2,500 g for about 5 minutes. After the initial CMC/trehalose layer, another 50 μl CMC-hydrogel can be layered over the mold and centrifuged for about 4 hours at 2,500 g, to dry the microneedle components in the mold. At the end of a drying process, the CMC-microneedle arrays are separated from the molds, trimmed off from excess material at the edges, collected and stored at about 4° C. The production molds may be cleaned and reused for further casting of microneedle arrays.

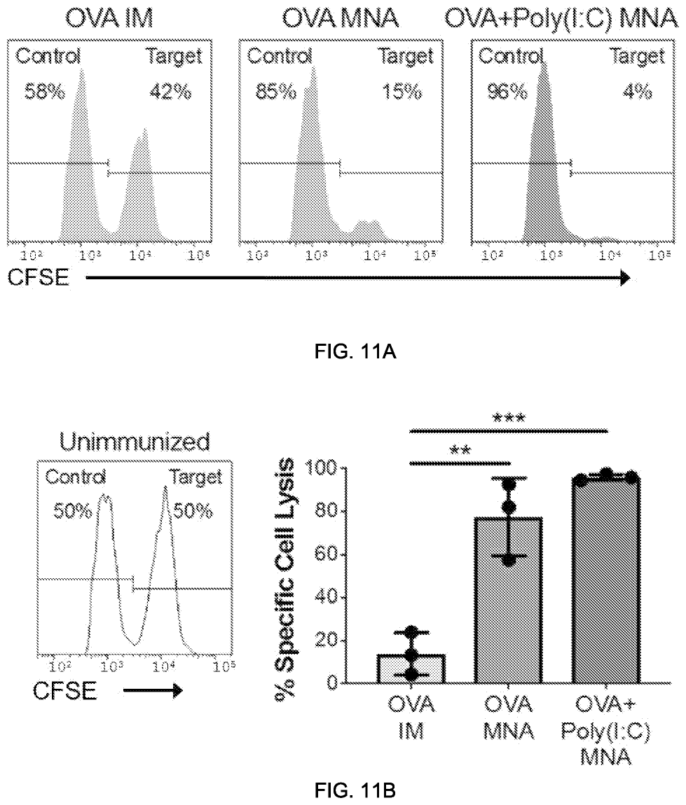

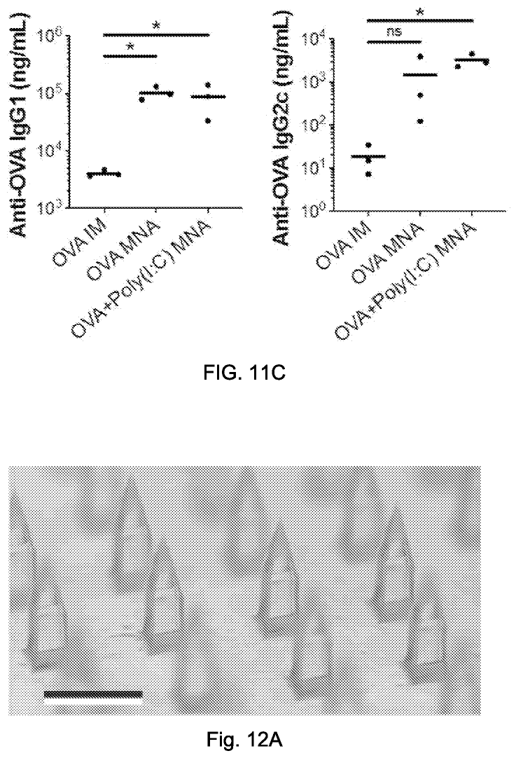

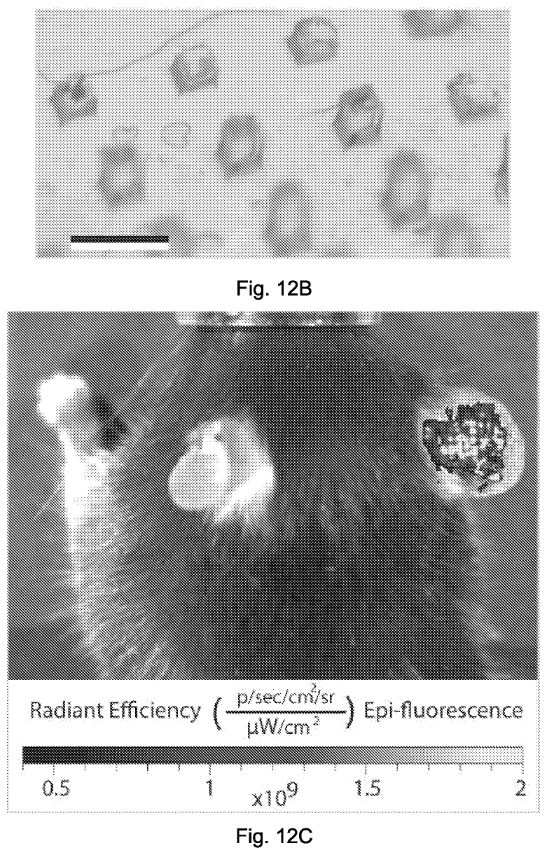

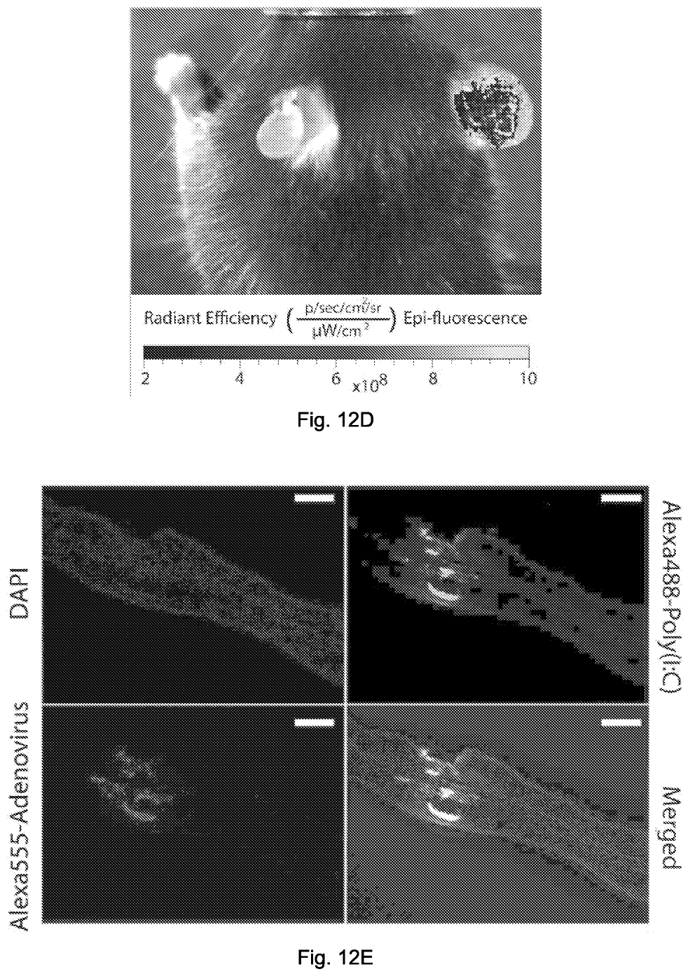

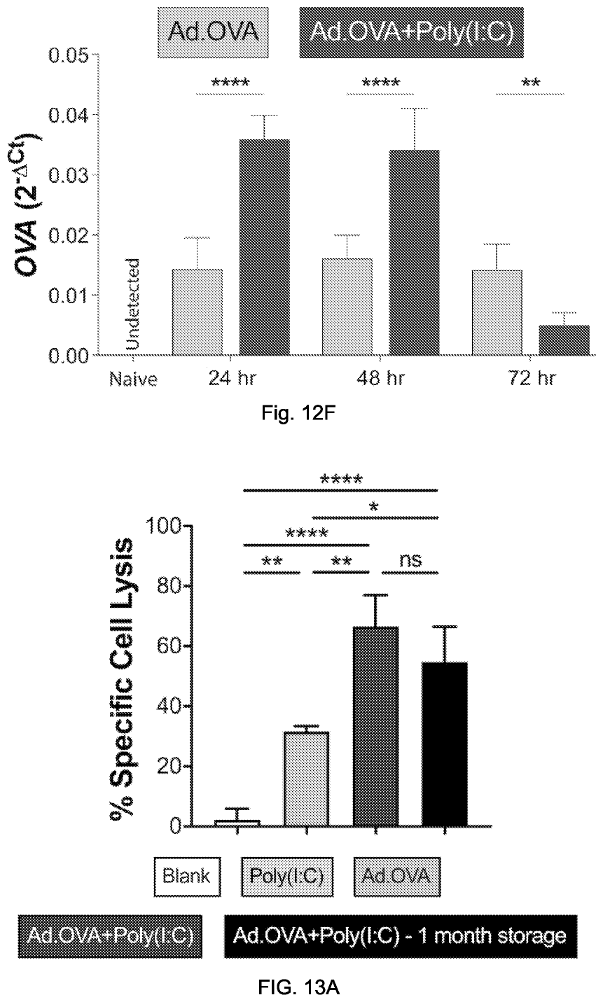

The solids in the microneedle may be formed with layers that do not contain active components and layers that contain active components. FIGS. 11A-D of PCT Application No. PCT/US2016/057363, incorporated herein by reference illustrate CMC-solids with different shapes and embedded active cargos on an upper layer which becomes, after micromilling, the portions of the microneedle with the active components. FIGS. 12A and 12B of PCT/US2016/057363, also illustrate CMC-solids with different shapes, with FIG. 12B showing a square shape and FIG. 12B showing a rectangular shape. Both CMC solids can be milled to dimensions for further processing as described herein. It should be understood that the geometries are not intended to be limiting. Any geometry can be used with the adenovirus particles disclosed herein.

United States Patent Publication Nos. 2011/0098651; 2014/0350472; 2015/0126923, 2016/0271381, and U.S. Pat. No. 8,834,423, describe certain exemplary microneedle arrays and methods of making and using microneedle arrays. As an example, apparatuses and methods are described for fabricating dissolvable microneedle arrays using master molds formed by micromilling techniques. For example, microneedle arrays can be fabricated based on a mastermold (positive) to production mold (negative) to array (positive) methodology. Micromilling technology can be used to generate various micro-scale geometries on virtually any type of material, including metal, polymer, and ceramic parts. Micromilled mastermolds of various shapes and configurations can be effectively used to generate multiple identical female production molds. The female production molds can then be used to microcast various microneedle arrays. Direct micromilling of mastermolds can replace other exemplary microneedle array production methods that involve expensive, complex and equipment-sensitive SU-8 based lithography or laser etching techniques, which are conventionally used to create mastermolds for dissolvable needle arrays. In addition, as discussed below, micromilling can provide for the construction of more complex mastermold features than can conventional lithography and laser etching processes. Precision-micromilling systems can be used for fabricating a microneedle mastermold, using micro-scale (for example, as small as 10 μm (micrometers or microns)) milling tools within precision computer controlled miniature machine-tool platforms. The system can include a microscope to view the surface of the workpiece that is being cut by the micro-tool. The micro-tool can be rotated at ultra-high speeds (200,000 rpm) to cut the workpiece to create the desired shapes. Micromilling process can be used to create complex geometric features with many kinds of material, which are not possible using conventional lithographic or laser etching processes. Various types of tooling can be used in the micromilling process, including, for example, carbide micro-tools or diamond tools.

Mastermolds can be micromilled from various materials, including, for example, Cirlex® (DuPont, Kapton® polyimide). Mastermolds can be used to fabricate flexible production molds from a suitable material, such as a silicone elastomer, e.g., SYLGARD® 184 (Dow Corning). The mastermold is desirably formed of a material that is capable of being reused so that a single mastermold can be repeatedly used to fabricate a large number of production molds. Similarly each production mold is desirably able to fabricate multiple microneedle arrays.

In one example, production molds are made from SYLGARD® 184 (Dow Corning), and are mixed at a 10:1 SYLGARD® to curing agent ratio. The mixture is degassed for about 10 minutes and poured over the mastermold to form an approximately 8 mm layer, subsequently degassed again for about 30 minutes and cured at 85° C. for 45 minutes. After cooling down to room temperature, the mastermold is separated from the cured silicone, and the silicone production mold is trimmed. From a single mastermold, a large number of production molds (e.g., 100 or more) can be produced with very little, if any, apparent deterioration of the Cirlex® or acrylic mastermolds.

In one example, to construct the microneedle arrays, a base material is used to form portions of each microneedle that have bioactive components and portions that do not. Of course, if desired, each microneedle can comprise only portions that contain bioactive components; however, to control the delivery of the bioactive component(s) and to control the cost of the microneedle arrays, each microneedle optionally is constructed such that a portion of the structure has a bioactive component and a portion does not include a bioactive component. Variations in the size, shape and number of the microneedles, and location of the bioactive component(s) in the microneedles, may be readily varied by varying the mastermold, or by varying the deposition and patterning of the materials used to produce the microarray.

A large variety of materials useful for preparation of the microneedle array are available, along with variation in the location of such materials in the microarray. Precise positioning and layering of the materials during, e.g., spin casting, of the microneedle array will yield any desired structure.

The microneedle array, both base and needles, may be manufactured from a single carrier composition including a dissolvable composition comprising trehalose and optionally carboxymethylcellulose, and an adenovirus particle. The “carrier composition” is one or more dissolvable and/or bioerodible compounds or compositions into which a bioactive agent is mixed, and in the context of the present disclosure forms a structure with physical parameters, and lack of negative effects on the bioactive agent as used herein, including sufficient safety to a patient, such that the carrier composition is useful as a component of the microneedles and microneedle arrays described herein.

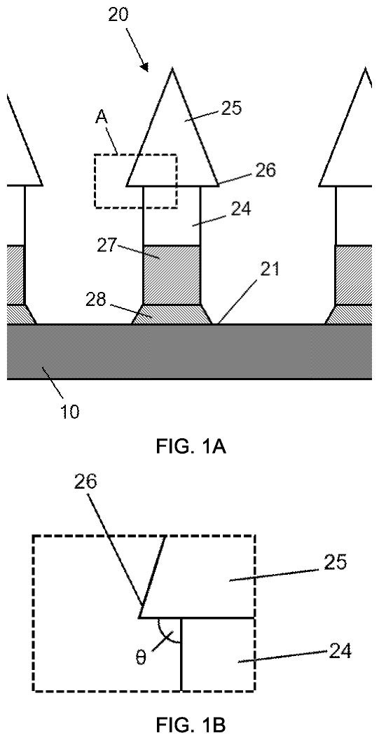

FIGS. 1A and 1B depict a microneedle array 10, in part, having a microneedle 20 and a backing layer 21. Additional, adjacent microneedles are partially depicted. The microneedle 20 comprises a stem 24 and a head 25. The head 25 comprises a ridge 26 extending beyond the peripheral boundary, e.g., circumference, of the stem 24, and as such, the head 25 may be described as undercut or barbed. The head 25, and optionally the stem 24, may comprise a bioactive or therapeutic agent such as the immunogen described herein mixed into, co-deposited in the mold with, absorbed into, or adsorbed onto the microneedle. The stem 24 may comprise a single material, which may be identical to the material of the head 25, or may be different than the material of the head 25. Although the stem 24 may be manufactured from a single material, it may comprise a dissolvable portion 27 that dissolves faster than the material of the head 25, e.g., within seconds under physiological conditions. The dissolvable portion 27 of the stem 24 may comprise a sugar or low molecular-weight dimer, oligomer, or polymer. In use, the microneedle 20 penetrates a patient's skin and the dissolvable portion rapidly-dissolves, either fully releasing the remainder of the microneedle in the patient's skin, or rendering that portion of the stem 24 sufficiently frangible, such that by pulling the backing layer 21 away from the patient's skin, and assisted by the undercutting of the head 25, the heads 25 of the microneedles, and any slower-dissolving portions of the stem 24, as compared to the dissolvable portion 27, will remain in the patient's skin. In embodiments of the microneedle array 10 where the stem 24 does not comprise a dissolvable portion 27, meaning the dissolvable portion 27 is the same material as the stem 24, the stem 24 still remains or is rendered frangible and may be retained in the patient's skin by dissolving and/or breaking of the stem 24.

In use, the microneedles 20 of the microneedle array 10 are pressed into the skin of a patient, and are left in place for a sufficient time to either inoculate the patient, or for the stems 24 and/or the dissolvable portions 27 of the microneedles to be dissolved or rendered sufficiently frangible so that removal of the backing layer would release the heads 25 of the microneedles 20 into the skin of the patient, such that the therapeutic agent(s), such as the immunogen, contained in and/or on the microneedles, is released into the skin of the patient. To prevent breaking off of the microneedles 20 on the patient's skin, prior to piercing of the skin with the microneedles 20, the base of the stem 24 of the microneedles may be reinforced, such as by use of a filleted base 28 as shown in FIG. 1A.

FIG. 1B depicts an enlargement of portion A, within the dashed rectangle as shown in FIG. 1A. Stem 24, head 25, and ridge 26 are depicted. FIG. 1B depicts the ridge 26 extending at an angle θ from the stem 24, where θ is 90°. In practice, θ may vary from 90°, and may range, from 20° to 100°, from 45° to 100°, from 60° to 95°, or 90°±2°, such as, without limitation, 75°, 76°, 77°, 78°, 79°, 80°, 81°, 82°, 83°, 84°, 85°, 86°, 87°, 88°, 89°, 90, 91°, 92°, 93°, 94°, or 95°. It should be recognized that the undercutting of the head and the transition from the head to the stem may not be perfectly linear, and these angles represent an average or best fit of the relationship between the stem and the underside of the ridge. In aspects, the amount of undercutting will be dictated by the ability to manufacture such structures, for example, using the methods described herein, including the ability to remove the microneedles from the mold after spin-casting. As such, the angle of undercut may be 90°, or may approach 90°, such as 90°±1°, 90°±2, 90° 3°, 90±4°, or 90°±5.

The microneedle array, and elements thereof are depicted in FIGS. 1A and 1B are having specific geometries, such as a cylindrical stem 24 and dissolvable portion 27, a conical head 25, a ridge 26 or undercut extending at a 90° angle (perpendicularly) from the stem 24, and a triangle-profiled filleted base. It would be understood that these geometries are merely exemplary and one of ordinary skill could adapt different shape profiles and still include the structure depicted in FIGS. 1A and 1B, while retaining the functional aspects of those structures.

In treating a patient, administration of the adenovirus particles may be accomplished by single or multiple doses. The dose administered to a subject in the context of the present disclosure may be sufficient to induce a beneficial therapeutic response in a subject over time, or to inhibit or prevent infection with a pathogen. The dose required may vary from subject-to-subject depending on the species, age, weight and general condition of the subject, the severity of the infection being treated, the particular immunogenic composition being used, and its mode of administration. An appropriate dose may be determined empirically. Multiple-size microneedle devices may be manufactured, or the microneedle devices may be manufactured such that the device can be divided, e.g., in halves, thirds, quarters, etc. by a practitioner or pharmacist to titer dosage for an individual.

For vaccinations, repeated immunizations may be necessary to produce an immune response in a subject. When administered in multiple doses, the booster doses are administered at various time intervals, such as weeks or months to years. In other examples, the microneedle device for expression of an immunogen are used as a booster following administration of one or more initial (prime) vaccination. As such, a prime boost strategy may be utilized. For example, a boost dose is administered about 14, 30, 60, 90, or more days after administration of the prime dose. Additional boosters can be administered at subsequent time points, if determined to be necessary or beneficial. Immunization protocols (such as amount of immunogen, number of doses and timing of administration) may be determined experimentally, for example by using animal models (such as mice or non-human primates), followed by clinical testing in humans.

For vaccination, initial injections may range from about 1 μg to about 1 mg, with some embodiments having a range of about 10 μg to about 800 μg, or from about 25 μg to about 500 μg. Following an initial administration of the immune stimulatory composition, subjects may receive one or several booster administrations, adequately spaced. Booster administrations may range from about 1 μg to about 1 mg, with other embodiments having a range of about 10 μg to about 750 μg, and still others a range of about 50 μg to about 500 μg. From 1 to 10¹⁴ viral particles or Us may be administered in the initial injection and/or booster, e.g., 1, 10, 10², 10³, 10⁴, 10⁵, 10⁶, 10⁷, 10⁸, 10⁹, 10¹⁰, 10¹¹, 10¹², 10¹³, or 10¹⁴ viral particles or Us per microneedle, or any increment therebetween. Periodic boosters at intervals of 1-5 years, for instance three years, may be desirable to maintain the desired levels of protective immunity.

Following immunization, the immune response may be assessed. For example, a biological sample may be obtained from the subject, and antibodies and/or reactive T cells specific for the target pathogen, can be assessed.

The following examples are provided to illustrate certain particular features and/or embodiments. These examples should not be construed to limit the disclosure to the particular features or embodiments described.

### Example 1—Fabrication and Testing of Microneedle Array

The skin is an attractive tissue target for vaccination, as it is readily accessible and contains a dense population of antigen-presenting and immune-accessory cells. Microneedle arrays (MNAs) are emerging as an effective tool for in situ engineering of the cutaneous microenvironment to enable diverse immunization strategies. Here, novel dissolving undercut MNAs and demonstrate their application for effective multicomponent cutaneous vaccination are presented. The MNAs are composed of micron-scale needles featuring pyramidal heads supported by undercut stem regions with filleted bases to ensure successful skin penetration and retention during application. Prior efforts to fabricate dissolving undercut microstructures were limited and required complex and lengthy processing and assembly steps. In the current study, we strategically combine three-dimensional (3D) laser lithography, an emerging micro-additive manufacturing method with unique geometric capabilities and nanoscale resolution, and micromolding with favorable materials. This approach enables reproducible production of dissolving MNAs with undercut microneedles that can be tip-loaded with multiple biocargos, such as antigen (ovalbumin) and adjuvant (Poly(I:C)). The resulting MNAs fulfill the geometric (sharp tips and smooth edges) and mechanical-strength requirements for failure-free penetration of human and murine skin to simultaneously deliver multicomponent (antigen plus adjuvant) vaccines to the same cutaneous microenvironment. Cutaneous vaccination of mice using these MNAs induces more potent antigen-specific cellular and humoral immune responses than those elicited by traditional intramuscular injection. Together, the unique geometric features of these undercut MNAs and the associated manufacturing strategy, which is compatible with diverse drugs and biologics, could enable a broad range of non-cutaneous and cutaneous drug delivery applications, including multicomponent vaccination.

### Materials and Methods

Ovalbumin (OVA; #A5503), polyinosinic-polycytidylic acid sodium salt (Poly(I:C); #P1530), carboxymethylcellulose (CMC, 90 kDa MW), D-(+)-trehalose dihydrate, polyvinylpyrrolidone (PVP, 40 kDa MW), polyvinyl alcohol (PVA, 87-90% hydrolyzed, 30-70 kDa MW), Allura Red AC (R40 dye), doxorubicin, 4,4′,5,5′-tetramethylbenzidine (TMB) peroxidase substrate, carbonate-bicarbonate buffer (pH 9.6), and Tween20 were purchased from Sigma-Aldrich (St. Louis, Mo.). Polydimethylsiloxane (PDMS) SYLGARD® 184 and VeroWhiteplus-RGD835 UV-curable resin were obtained from Dow Corning (Midland, Mich.) and Stratasys (Eden Prairie, Minn.), respectively. Green fluorescent Degradex PLGA microspheres (10 μm diameter) were acquired from Phosphorex (Hopkinton, Mass.). Alexa 555-labeled OVA (Invitrogen), Alexa 680-labeled OVA (Invitrogen), Texas Red-labeled dextran (40 kDa MW; Invitrogen), Pierce Micro BCA Protein Assay Kit, SYBR Green EMSA nucleic acid stain, endotoxin-free HyClone Cell Culture Grade Water, RNase-free Ambion TE Buffer (pH 8.0), carboxyfluorescein succinimidyl ester (CFSE; Invitrogen), and DAPI were purchased from Thermo Fisher Scientific (Waltham, Mass.). Anti-OVA IgG1 (Cayman Chemical, Ann Arbor, Mich.), anti-OVA IgG2c (Chondrex, Redmond, Wash.), normal goat serum and biotinylated goat anti-mouse IgG1 and IgG2c secondary antibodies (Jackson ImmunoResearch, West Grove, Pa.), streptavidin-HRP (BD Biosciences, San Jose, Calif.), and OVA257-264 (SIINFEKL) peptide (Anaspec, Fremont, Calif.) were used for immune assays.

### Fabrication of Dissolving Microneedle Arrays

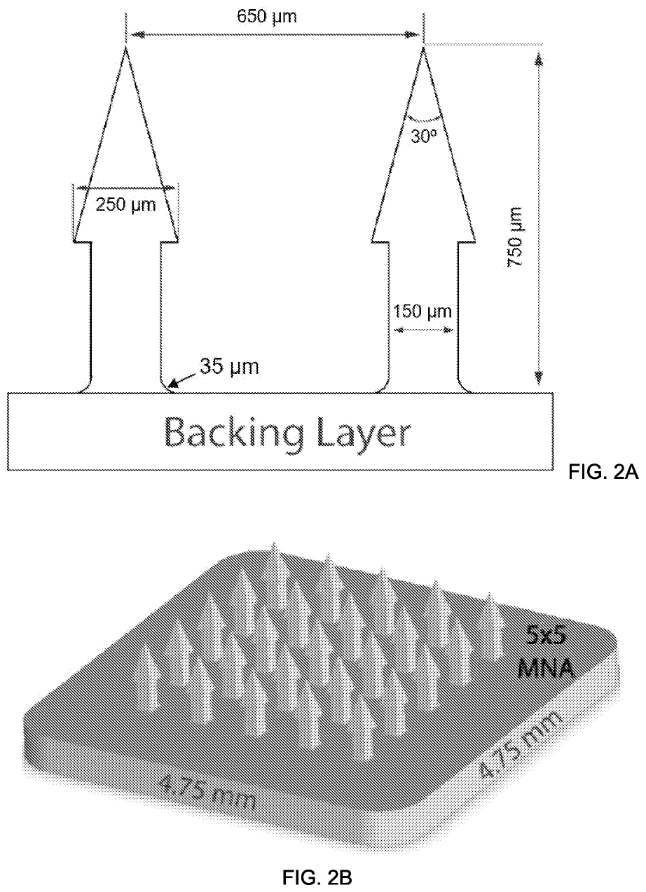

Microneedle and array designs: The unique microneedle array (MNA) design utilized in this study is shown in FIGS. 2A-2B. This particular microneedle design consisted of a sharp-tipped pyramid head and an undercut stem portion with a filleted base. The microneedle was 750 μm in height with a 30° apex angle. The stem portion of the microneedle was 150 μm in width and extended from the bottom of a square pyramid head (250 μm×250 μm base area) to the backing layer of MNA with a 35 μm radius filleted connection. The fillet was specifically designed at the microneedle base to avoid sharp corners and associated mechanical stress concentration, considerably increasing microneedle strength performance during manufacturing processes and skin insertion (Bediz et al., “Dissolvable microneedle arrays for intradermal delivery of biologics: fabrication and application”, 2014, Pharm. Res., 31:117-135; Rad et al. “High-fidelity replication of thermoplastic microneedles with open microfluidic channels, 2017, Microsyst. Nanoeng., 3: 17034). The apex angle, width, and height of the microneedles were chosen based on skin anatomy and skin insertion mechanics to ensure failure-free penetration (Bediz et al.; Prausnitz, M. R. “Engineering Microneedle Patches for Vaccination and Drug Delivery to Skin”, 2017, Annu Rev Chem Biomol Eng; 8:177-200). Notably, this design introduces a novel undercut, or anchor feature, which improves skin retention during application, but still allows direct removal of MNAs from flexible production molds throughout the manufacturing process. The tip-to-tip distance between microneedles in the 5×5 arrays was 650 μm, and the size of MNA was 4.75 mm×4.75 mm. The array design (microneedle spacing) was based on solid mechanics considerations and skin insertion mechanics to avoid a “bed of nails” effect during skin penetration (Bediz et al.; Korkmaz et al., “Therapeutic intradermal delivery of tumor necrosis factor-alpha antibodies using tip-loaded dissolvable microneedle arrays”, 2015, Acta Biomater., 24:96-105; Verbaan et al., “Improved piercing of microneedle arrays in dermatomed human skin by an impact insertion method”, 2008, J. Control. Release, 128:80-88). The three-dimensional micro-additive manufacturing (3D-μAM) approach provides a simple, reproducible, and revolutionary means to produce the proposed unique MNA design from a 3D-CAD drawing, and allows individuals with no microfabrication expertise to easily create a broad range of MNA designs.

### Manufacturing Strategy

The manufacturing strategy used to fabricate dissolving MNAs with novel microneedle designs is graphically summarized in FIG. 3. This strategic six-step approach exploits μAM and micromolding to create dissolving undercut MNAs, while simultaneously achieving high-throughput fabrication: (1) 3D-CAD drawing of the MNA design; (2) direct production of a master MNA from the CAD drawing by 3D direct laser writing using a non-dissolvable resin (IP-S); (3) high-fidelity replication of master MNA with UV-curable resin (VeroWhite) by micromolding; (4) creation of MNA master molds that consist of multiple master MNA replicas on 3D-printed MNA holders; (5) manufacturing of elastomer (PDMS) MNA production molds by micromolding; and (6) fabrication of tip-loaded, dissolving MNAs with undercut microneedles incorporating a vaccine or other biocargo in a water-soluble biocompatible material (e.g., carboxymethylcellulose (CMC) and trehalose) through a spin-casting method. The last step of the process can be modified depending on the biocargo of interest, and typically involves spin-casting cargo (e.g., vaccine) into the tip of the PDMS production molds, followed by spin-casting a dissolvable hydrogel (e.g., CMC/trehalose) into the production molds to serve as the structural material. Notably, the master MNA, master molds consisting of multiple master MNA replicas, and elastomer production molds are reusable, reducing the fabrication costs for dissolving undercut MNAs. At each stage of the fabrication process, optical stereomicroscopy (ZEISS Stemi 2000-C microscope with Olympus OM-D E-M511 camera) was used to assess geometric integrity of the microneedles.

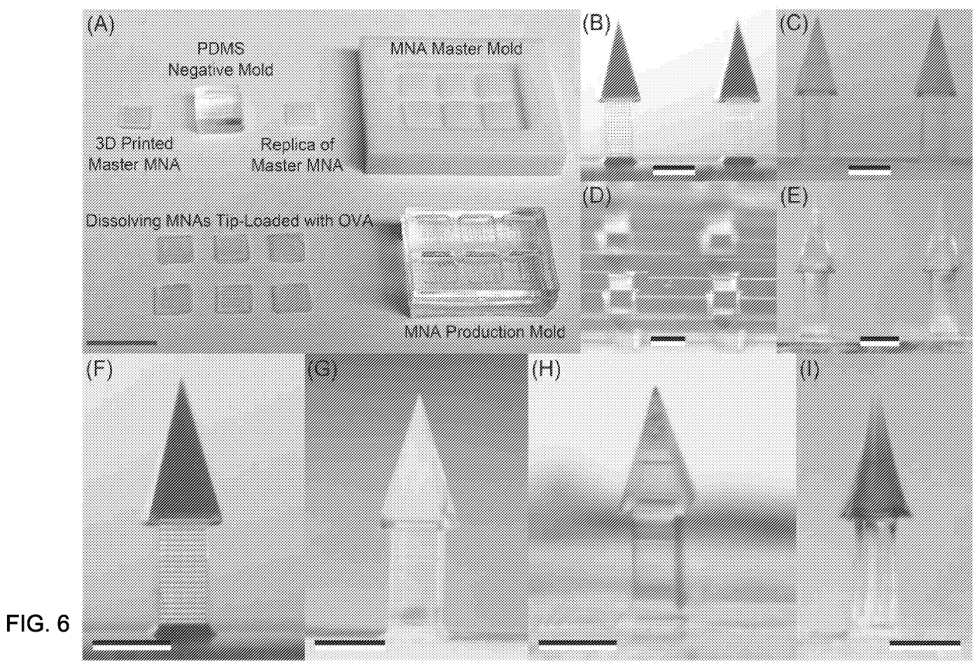

Fabrication of master MNA: The unique MNA geometry was designed in SolidWorks 2018 CAD software and directly created from the 3D-CAD drawing (FIGS. 6A-6B) using 3D laser printing (Nanoscribe Photonic Professional, GT; Nanoscribe Struensee, Germany) with the photopolymeric resist IP-S. The Nanoscribe printing system was equipped with a laser generator, an optical cabinet, a Zeiss optical microscope attached to a lens to focus the laser beam, a Galvo mirror system to direct the laser-beam scanning, a piezoelectric stage for precise motion control, and software (Nanowrite) to execute 3D printing. The whole system was placed on an optical table to eliminate vibrations during the printing process.

To fabricate the master MNA, the CAD design was converted into ‘STL’ (StereoLithography) format. The STL file was loaded into the specialized software (DeScribe, Germany) for the Nanoscribe system to select the processing conditions (distance of slicing, hatching, and splitting). Finally, the STL file was converted into ‘GWL’ (General Writing Lithography) format and exported to the Nanowrite software to print the master MNA. The master MNA was fabricated using Galvo-scan mode in XY plane and piezo-scan mode in Z direction. The master MNA was split into 220 μm×220 μm×200 μm blocks within the working range and then stitched together. Laser power and writing speed were set to 100 mW and 6 cm/s, respectively. Minimum and maximum slicing distances of 0.3 μm and 0.5 μm, respectively, were used. The master MNA was then printed through two-photon polymerization of the IP-S photoresist by a femtosecond pulsed laser at a wavelength of 750 nm using a unique deep-in-liquid mode with a 25×NA0.8 objective in Shell and Scaffold mode. After printing, the master MNA was developed in the photoresist solvent propylene glycol monomethyl ether acetate (PGMEA) for 30 min, followed by a 5 min isopropyl alcohol rinse. The master MNA was then air-dried and placed under UV light (365 nm, 16 mW/cm² intensity) for 30 min to further crosslink the body to make the master MNA structure strong.

Replication of Master MNA: A two-stage micromolding method was used to replicate the master MNA with high-fidelity using a UV-curable resin. First, an elastomer mold, which is a negative mold of the master MNA, was manufactured from polydimethylsiloxane (PDMS) by soft-lithography. Elastomer molding with PDMS is a well-established technique for rapid, accurate, and reproducible replication of high-fidelity micron-scale structures (Losic et al., “Rapid fabrication of micro- and nanoscale patterns by replica molding from diatom biosilica”, 2007, Adv. Funct. Mater., 17:2439-2446; Gates et al., “Replication of vertical features smaller than 2 nm by soft lithography”, 2003, J. Am. Chem. Soc., 125:14986-14987). Briefly, the master MNA was mounted in a petri-dish with a diameter of 5 cm, and PDMS was prepared using a two-component curable silicone elastomer, SYLGARD® 184 (10:1 base-to-curing agent). The PDMS was poured over the master MNA mounted in the petri-dish and degassed for 15 min. Next, the master MNA with degassed PDMS was cured at 70° C. for 1 h. The cured PDMS was cooled to room temperature for 5 min and then separated from the master MNA to obtain the negative PDMS mold.

The second processing step used the negative PDMS mold to fabricate positive master MNA replicas from a UV-curable resin (VeroWhiteplus-RGD835). For each PDMS mold, 20 μL of liquid resin was poured onto the molds, and then the molds were centrifuged (4500 RPM at 20° C. for 1 min; Thermo Fisher Scientific Sorvall Legend XTR centrifuge with Swinging Bucket Rotor TX-750) to fill the microneedle-shaped wells with resin. The resin was then treated under UV light (365 nm) with 21.7 mW/cm² intensity for 5 min from both the top and bottom to cure the base and the microneedle tips. To ensure the backing layers of the master MNA replicas were flat, an additional 50 μL of UV-curable resin, which exceeded the remaining volume available, was deposited onto the PDMS mold. A glass slide was placed on top of the mold to get rid of the excess resin, thereby creating a uniform flat surface at the base. The liquid resin was then cured from the top side for 5 min and demolded to obtain a replica of the master MNA.

Creation of MNA master molds and production molds: To improve productivity of the manufacturing process for dissolving MNAs, the MNA master molds were created by assembling six master MNA replicas onto MNA holders fabricated by Stratasys® from a non-dissolvable photo-polymer (VeroWhite) using a high-resolution Polyjet 3D printing system (Objet Connex 500 multi-material). A 3D model of the MNA holder was created using SolidWorks 2018 CAD software and then converted into the ‘STL’ (StereoLithography) file format. Subsequently, the specialized software (Objet Studio) sliced this 3D model into 2D cross-sectional layers, creating a computer file that was sent to the 3D printer system at Stratasys. Channels in the 3D printed MNA holder were designed to serve as pockets in the MNA production molds to assist as reservoirs for both the bioactive cargo (e.g., vaccine) and the structural hydrogel material of dissolving MNAs during the spin-casting process. The MNA master molds were baked at 80° C. overnight in a vacuum oven to facilitate effective molding of elastomer MNA production molds. Subsequently, MNA production molds that included microneedle-shaped wells for six MNAs were fabricated from PDMS as described for replication of the master MNA. Notably, a single MNA master mold can be used repeatedly to fabricate multiple PDMS production molds.

Production of dissolving MNAs: Dissolving MNAs with novel undercut microneedles tip-loaded with multicomponent vaccines (OVA antigen±Poly(I:C) adjuvant) were manufactured through a spin-casting technique with centrifugation at room temperature. First, 5 μL of an aqueous solution of OVA (25 mg/mL) was dispensed to each MNA reservoir on the PDMS production molds, and production molds were centrifuged (1 min at 4500 rpm) to fill the microneedle-shaped cavities. Excess OVA solution within the reservoir was then recovered, and production molds were centrifuged (30 min at 4500 rpm) to ensure that dry OVA cargo was located at the tip portion of the microneedle-shaped cavities in the production molds. For MNAs integrating OVA and Poly(I:C), the aforementioned process was repeated with 5 μL of an aqueous solution of Poly(I:C) (62.5 mg/mL). The final MNAs used for cutaneous vaccination experiments included 10 μg OVA and 25 μg Poly(I:C) per MNA. The tip-loading process and recovery of excess biocargo is depicted in FIG. 4.

After loading vaccine biocargo at the tips of microneedles, the MNA structural biomaterial was prepared by dissolving a 70:30 mixture of sodium carboxymethylcellulose (CMC) and D-(+)-trehalose dihydrate in endotoxin-free water at a total solute concentration of 30% w/w. The resulting CMC/trehalose hydrogel was loaded onto each MNA in the PDMS production molds (40 μL each) to fill the remaining volume of the microneedles and to form the MNA backing layer. Hydrogel-loaded production molds were centrifuged (5 h at 4500 rpm) to obtain the final dissolving undercut MNAs for cutaneous vaccination experiments. MNAs were then removed from production molds with tweezers, or forceps, by pulling two diagonal corners of the MNA base away from the mold. To demonstrate the broader material capabilities of our manufacturing strategy for dissolving MNAs with undercut microneedles, MNAs were also fabricated using a 40% w/w hydrogel with a 60:40 mixture of polyvinylpyrrolidone (PVP) and polyvinyl alcohol (PVA) through the spin-casting method.

### Quantification of Antigen and Adjuvant Loading

Microneedles were dissolved in TE Buffer, and concentrations of OVA and Poly(I:C) were measured using a Micro BCA protein assay and SYBR Green nucleic acid assay, respectively. Loading error, defined as the difference between measured and theoretical amounts of biocargo in microneedles as a percentage of the theoretical amount, was calculated. To determine loading efficiency, excess biocargo recovered from the MNA production mold reservoir after loading and prior to drying (FIG. 11 (C)) was quantified. Loading efficiency=[biocargo in microneedles/(biocargo loaded to mold—excess biocargo recovered)]×100%. Results are reported as mean±SD (N=6).

### Cutaneous Vaccine Delivery to Human Skin Explants Using MNAs

Preparation of ex vivo human skin explants: Human skin explants were prepared as described previously (Morelli et al., “CD4⁺ T cell responses elicited by different subsets of human skin migratory dendritic cells”, 2005, J. Immunol., 175:7905-7915). Briefly, normal human skin from deidentified healthy donors undergoing plastic surgery was acquired through the Pitt Biospecimen Core and used according to University of Pittsburgh Medical Center guidelines. Tissue was rinsed in 70% ethanol and then in phosphate-buffered saline (PBS). Human skin explants (approximately 1 mm thick) were harvested using a Silver's miniature skin graft knife (Padgett, Integra Miltex, Plainsboro, N.J.), and then cut into 20 mm×20 mm square pieces. The resulting human skin samples comprised epidermis and a thin layer of underlying dermis.

Imagining Analysis: To evaluate undercut MNA-directed intradermal biocargo (e.g., vaccine) delivery to living human skin explants, several imaging analyses were performed. Tip-loaded MNAs incorporating a red cargo (Allura Red R40 dye) were fabricated using the manufacturing strategy described above. Prior to application of MNAs to human skin explants, MNAs were imaged using an optical stereomicroscope. Subsequently, MNAs were applied to human skin explants and removed after 10 min. An optical stereomicroscope was then used to image the patterns of colored biocargo deposited from MNAs into the human skin. Remaining MNA materials after application were also imaged. For further qualitative assessment of MNA-directed intradermal vaccine delivery to human skin, MNAs containing both Alexa555-labeled OVA and Alexa488-labeled Poly(I:C) were fabricated, applied to human skin explants for 10 min, and removed. Targeted areas of the human skin explants were fixed in 2% paraformaldehyde, cryopreserved with 30% sucrose solution, flash frozen in optimum cutting temperature (OCT) compound, and cryo-sectioned into 10 μm thick sections. Human skin cross-sections were counter-stained with a fluorescent nuclear dye (DAPI) and imaged using a bright-field and epifluorescence microscope (Nikon Eclipse E800) to detect Alexa555-OVA and Alexa488-Poly(I:C), with bright-field images taken to better visualize the stratum corneum breaching.

### MNA-Directed Skin Immunization In Vivo

Mice: Female C57BL/6J mice were purchased from The Jackson Laboratory (Bar Harbor, Me.) and used at 8-10 weeks of age. Mice were maintained under specific pathogen-free conditions at the University of Pittsburgh, and all experiments were conducted in accordance with the institutional animal care and use committee (IACUC) guidelines.

Quantification of antigen and adjuvant delivery with MNAs: OVA+Poly(I:C) MNAs were applied to murine abdominal skin for 5, 10, or 20 min, and then remaining MNAs were removed and dissolved in TE Buffer. Concentrations of OVA and Poly(I:C) were measured using a Micro BCA protein assay and SYBR Green nucleic acid assay, respectively. Quantities of OVA and Poly(I:C) delivered to skin were calculated by subtracting the amount remaining from the mean amount loaded, and delivery is reported as percentage of the initial amount loaded (mean±SD, N=6).

In vivo IVIS imaging: In vivo intradermal vaccine delivery with dissolving undercut MNAs was demonstrated on a C57BL/6J mouse. Tip-loaded CMC/trehalose MNAs integrating both Alexa555-labeled OVA and Alexa488-labeled Poly(I:C) were created using the manufacturing strategy described above, applied to the abdomen of an anesthetized mouse for 10 min, and then removed. Fluorescent OVA+Poly(I:C) MNAs were imaged before and after in vivo application by optical stereomicroscopy and epifluorescence microscopy. To show delivery of the fluorescent multicomponent vaccine, the mouse was imaged with an IVIS 200 in vivo imaging system (PerkinElmer, Waltham, Mass.), using the corresponding filters to detect Alexa488-Poly(I:C) and Alexa555-OVA at the MNA application site. Images were then post-processed using Living Image software (PerkinElmer).

Cell-mediated and humoral immune responses: Mice were immunized by application of 10 μg OVA±25 μg Poly(I:C) MNAs to the right and left sides of abdomen (two MNAs per mouse) or by two intramuscular injections of 10 μg OVA in PBS into the hindlimb gastrocnemius muscles. Control mice were left untreated (i.e., naïve), or treated with blank MNAs (without antigen or adjuvant). Cutaneous or intramuscular immunizations were repeated 7 days later. In vivo OVA-specific cytotoxic T-cell activity and OVA-specific antibody responses were evaluated 5 days after the second immunization (booster dose) using well-established techniques (Morelli et al.; Condon et al. “DNA-based immunization by in vivo transfection of dendritic cells”, 1996, Nat. Med., 2: 1122-1128).

For OVA-specific antibody responses, blood was collected from anesthetized mice at the time of sacrifice by cardiac puncture, and serum was isolated using BD Microtainer serum separator tubes (BD Biosciences, San Jose, Calif.). OVA-specific IgG1 and IgG2c antibodies in serum were measured by indirect ELISAs. Costar EIA/RIA plates (Corning Inc., Corning, N.Y.) were coated with OVA (100 μg/mL in 0.5 M carbonate-bicarbonate buffer) by overnight incubation at 4° C. Plates were washed (3×) with 0.05% Tween20 in PBS, and blocked with 1% goat serum in PBS for 1 h at 37° C. Serum samples and standards (anti-OVA IgG1 or anti-OVA IgG2c) were diluted with 1% goat serum, added to plates, and incubated 2 h at 37° C. After washing (3×), plates were incubated for 1 h at 37° C. with biotinylated secondary antibodies (goat anti-mouse IgG1 or IgG2c, 1:20,000 in 1% goat serum). Plates were then washed (3×) and incubated for 30 min with streptavidin-HRP (1:1000 in 1% goat serum). Plates were washed (3×) again and incubated at room temperature with TMB peroxidase substrate for 2-3 min, and the reaction quenched with 1.0 M H₂SO₄. For all ELISAs, absorbance at 450 nm (OD450) was read with a SpectraMax 340PC plate reader (Molecular Devices, Sunnyvale, Calif.), and serum concentrations calculated from standard curves.

To assess OVA-specific cytotoxic T-cell (CTL) activity, splenocytes from naïve mice were pulsed with 2 μg/mL OVA257-264 (SIINFEKL) peptide, or left unpulsed for 1 h. Antigen pulsed splenocytes were washed and stained with high concentration CFSE (10 μM), while unpulsed splenocytes were labeled with low concentration CFSE (1 μM) for 15 min at 37° C. A 1:1 mixture of pulsed target cells and unpulsed control cells (107 each) was intravenously (IV) injected into immunized and naïve mice. Twenty hours after adoptive transfer, spleens of mice were isolated, and killing of target cells was evaluated by comparison of the antigen pulsed and unpulsed populations by flow cytometry to quantify OVA-specific killing of the high CFSE labeled SIINFEKL-pulsed targets. Specific lysis was calculated and expressed as a percentage of maximum lysis as: % Lysis={1−[(mean CFSE^{low}/CFSE^{high} ratio from naïve mice)/(CFSE^{low}/CFSE^{high} ratio from vaccinated mouse)]}×100%.

Statistical analyses: Statistical analyses were performed using GraphPad Prism v8 (San Diego, Calif.). Data from vaccination experiments were analyzed by one-way independent ANOVA, followed by Tukey's or Dunnett's post-hoc testing. Differences were considered significant if p<0.05.

### Results and Discussion

Fabrication of dissolving undercut microneedle arrays: Penetration, dissolution, and delivery efficiency are parameters relevant to dissolving MNA-mediated cutaneous immunization. These factors depend on MNA design, and microneedle geometry contributes to the success of MNA-based cutaneous drug delivery. A number of different microneedle geometries such as circular, obelisk, and pyramid microneedles have been used for MNA-directed intradermal drug delivery. MNAs with obelisk microneedle geometries may result in better penetration and cutaneous delivery efficiency as compared to those with prevailing pyramid microneedles. Further, localizing biocargo to the skin-penetrating tip portion of the microneedles enhances delivery efficiency. Maximizing intradermal delivery efficiency is particularly important for effective cutaneous immunization to enable skin microenvironment conditioning while minimizing the necessary quantities of expensive vaccine components. These factors were considered when developing novel MNAs and the associated manufacturing strategy.

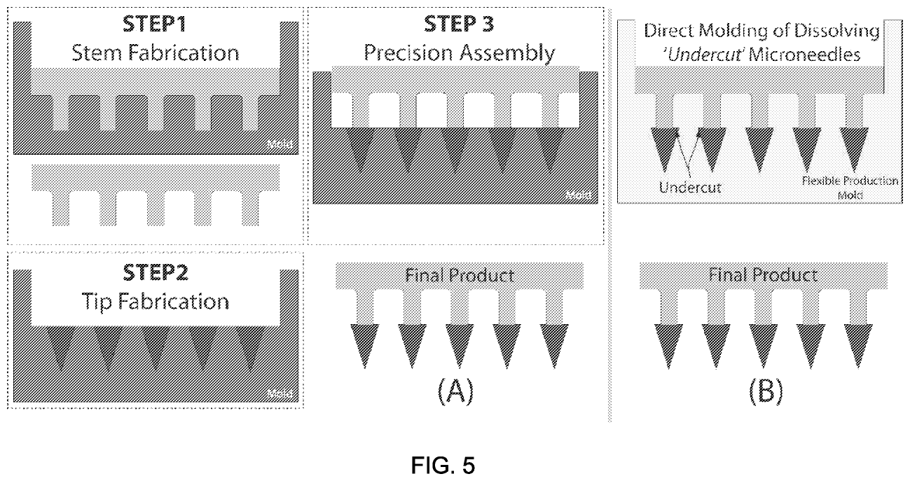

Dissolving microneedles for cutaneous vaccination are provided, which feature undercut geometry and biomolecules localized in the needle apex. This undercut microneedle geometry is believed to result in better retention and other practical advantages in skin and non-cutaneous tissues. Undercut microneedles present complex fabrication challenges (e.g., summarized in FIG. 5). Micromolding is a useful method for high-throughput manufacturing of microstructures; however, fabrication of microstructures with undercut features through micromolding requires complex processing steps and precision assembly of separately molded, or machined, microneedle tips and shafts. Here, we present a manufacturing strategy and materials to fabricate dissolving MNAs with undercut features (FIGS. 2A-2B) through micromolding (FIG. 3), eliminating complicated engineering procedures. Importantly, and counterintuitively, we show that undercut microneedles can be directly removed from the flexible production molds which are reusable for several processing cycles—substantially improving cost and productivity. These results suggest that during the micromolding processes, MNA production molds undergo elastic deflection without permanent deformation, and the mechanical stress distribution (caused by removal forces) is smaller than the strength of the microneedle materials, resulting in failure-free removal of undercut microneedles.

The manufacturing strategy we utilize uniquely enables reproducible fabrication of high-quality, tip-loaded dissolving MNAs with undercut features from different and widely-used dissolving microneedle biomaterials, including CMC/trehalose and PVP/PVA compositions (See, e.g., Bediz et al.; Lee et al. “Dissolving microneedles for transdermal drug delivery”, 2008, Biomaterials, 29:2113-2124; Korkmaz et al.). The manufacturing and processing steps schematically depicted in FIG. 3 result in the final products shown in FIG. 4 (A). Specifically, the master MNA was fabricated from IP-S photoresist by 3D direct laser writing. IP-S is a specific material designed for 3D laser lithography and provides high resolution and mechanical integrity for micro- and nano-structures. We find that 3D laser lithography based on two-photon polymerization provides an effective means for fabrication of undercut MNA designs with smooth edges and sharp tips (˜2 μm tip radius), and without any unwanted residues (e.g., machining chips) (FIG. 6 (A, F)). To enable more rapid, parallel fabrication of dissolving MNAs, the master MNA was replicated through a two-step micromolding process (FIG. 6 (C, G)). The IP-S master MNA was used to fabricate a flexible PDMS mold through soft-lithography, and the resulting PDMS molds were used to manufacture several VeroWhite MNA replicas through UV-curable micromolding. PDMS is a commonly used elastomer with tunable flexibility and low cost for molding of micro- and nano-structures (Lee et al.; Losic et al.). VeroWhite resin is a wear-resistant, acrylic-based photo-polymer extensively used for 3D Polyjet printers (Lin et al., “3D printed, bio-inspired prototypes and analytical models for structured suture interfaces with geometrically-tuned deformation and failure behavior”, 2014, J. Mech. Phys. Solids, 73:166-182), which renders it an ideal material for MNA master molds. Six MNA replicas were then assembled into one MNA master mold, and this master mold was used to produce several PDMS MNA production molds (FIG. 6 (D)). Collectively, these processing steps, along with high geometric capability of 3D direct laser writing, resulted in an effective MNA manufacturing strategy. Furthermore, rapid replication of the 3D printed master MNA using a wear-resistant moldable material improved productivity. Based on these results, it is believed that other undercut features could be achieved with this versatile approach, potentially using materials with different Young's modulus for the elastomer molds or/and polymers with different strength properties for microneedles.

Upon fabrication of the MNA master molds with six MNA replicas, dissolving MNAs that integrate vaccine components in the tip portion of the microneedles were fabricated using the conventional three-stage manufacturing strategy through master mold to production mold to final dissolving MNAs (Bediz et al.; Lee et al.). Dissolving MNAs that incorporated the vaccine components (OVA±Poly(I:C)) in the tip portion of the undercut microneedles were fabricated through the spin-casting process (FIG. 6 (E)). Total OVA and Poly(I:C) content in microneedles was determined to be 10.15±0.87 μg and 24.29±1.60 μg, respectively. With nominal doses of 10 μg OVA and 25 μg Poly(I:C), average loading errors were 5.8% and 6.1%, respectively. During the microneedle tip-loading process, recovery of excess biocargo from MNA production mold reservoirs prior to drying (detailed in FIG. 4) reduces biocargo waste and enables a higher loading efficiency of 77.8±5.8%, which is especially important when working with more expensive vaccine components. For vaccine experiments, we used MNAs made of CMC and trehalose, two FDA-designated “Generally Recognized as Safe” (GRAS) biomaterials. The water-solubility and mechanical strength of CMC make it a good structural material for MNAs (Lee et al.), while trehalose is a disaccharide known to enhance stability of proteins (Kaushik et al., “Why is trehalose an exceptional protein stabilizer? An analysis of the thermal stability of proteins in the presence of the compatible osmolyte trehalose”, 2003, J. Biol. Chem., 278:26458-26465).

To demonstrate compatibility of our MNA fabrication process and the undercut microneedle geometry with another dissolvable biomaterial composition commonly used in the MNA field, we fabricated some MNAs using a PVP/PVA hydrogel (FIG. 6 (H)). Additionally, MNAs with undercut microneedles tip-loaded with a red colored model drug (doxorubicin) were fabricated to facilitate imaging and demonstrate compatibility of the fabrication process with small molecule agents (FIG. 6 (1)). Demonstrated compatibility of the MNA fabrication process and undercut microneedle geometry with different types of cargos and material compositions makes application-driven optimization possible, as materials can be selected based on compatibility with bioactive cargo, dissolution requirements, and/or necessary mechanical properties for insertion into different types of skin (e.g., normal skin vs. psoriatic plaques).

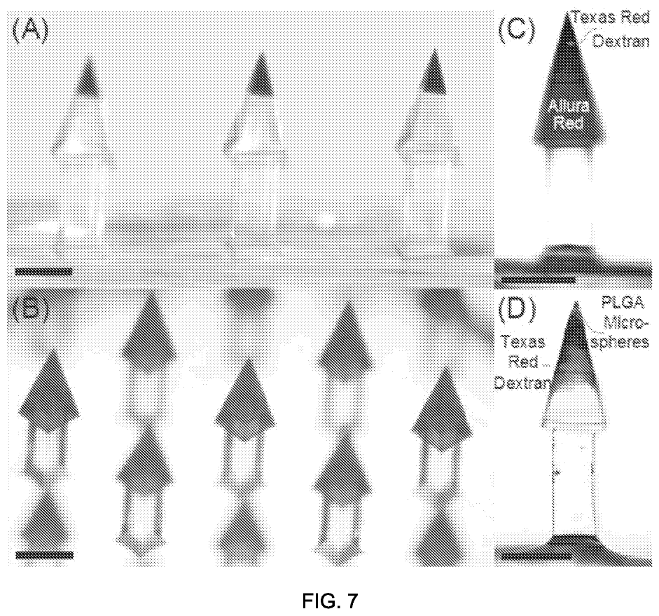

In addition to cutaneous vaccination, these dissolving undercut MNAs can be used for a broad range of intradermal and non-cutaneous (e.g., liver, ocular, and cardiac tissues) drug delivery applications (Li et al., “Rapidly separable microneedle patch for the sustained release of a contraceptive”, 2019, Nat. Biomed. Eng., 3: 220-229; Than et al., “Self-implantable double-layered micro-drug-reservoirs for efficient and controlled ocular drug delivery”, 2018, Nat. Commun., 9: 4433; Tang et al., “Cardiac cell-integrated microneedle patch for treating myocardial infarction”, 2018, Sci. Adv., 4:eaat9365). Through the spin-casting process, biocargo(s) of interest can be located either at the tips of microneedles (FIG. 7 (A)), or throughout the entire pyramid region (FIG. 7 (B)), depending on dose requirements. Furthermore, a number of sequential spin-casting steps can be performed to fabricate high-quality MNAs with undercut microneedles that incorporate multiple cargos in their pyramid regions (FIGS. 7 (C) and 7 (D)). As such, the presented approach and novel MNA designs are compatible with single and combination therapies for several cutaneous and non-cutaneous applications. Importantly, same production molds can be re-used to fabricate dissolving MNAs, suggesting that removal of undercut MNAs from flexible PDMS production molds results in elastic deformation for several process cycles without destroying the production molds. For example, the dissolving MNAs in FIGS. 7 (A) and 7 (C) were obtained using the same PDMS production mold at the first and twelfth cycles, respectively. Furthermore, we are currently capable of fabricating 5000+ MNAs per day in our laboratories, and these fabrication processes can be scaled up using industrial grade manufacturing strategies.

Additive manufacturing (AM), or 3D printing, has proven useful for the preparation of drug delivery systems (Prasad et al., “3D printing technologies for drug delivery: a review”, 2016, Drug Dev. Ind. Pharm., 42:1019-1031; Jonathan et al., “3D printing in pharmaceutics: a new tool for designing customized drug delivery systems”, 2016, Int. J. Pharm., 499:376-394). Indeed, there are currently FDA approved, 3D-printed drug delivery systems, such as Spritam® tablets (Prasad et al.; Jonathan et al.). A unique advantage of AM over traditional subtractive fabrication techniques is the possibility for accurate and reproducible manufacturing of 3D complex geometries without design limitations (Johnson et al.). As such, AM offers a high degree of design flexibility and control, and thus enables rapid design-to-fabrication turnaround for optimal application-driven drug delivery systems (Economidou et al., “3D printing applications for transdermal drug delivery”, Int. J. Pharm., 2018, 544: 415-424). Indeed, micro-scale AM has been effectively used for accurate and reliable fabrication of intradermal drug delivery systems (Johnson et al.). However, the unique advantages of AM have yet to be exploited for scalable fabrication of high-quality dissolving MNAs with novel designs for a broad range of drug delivery applications.

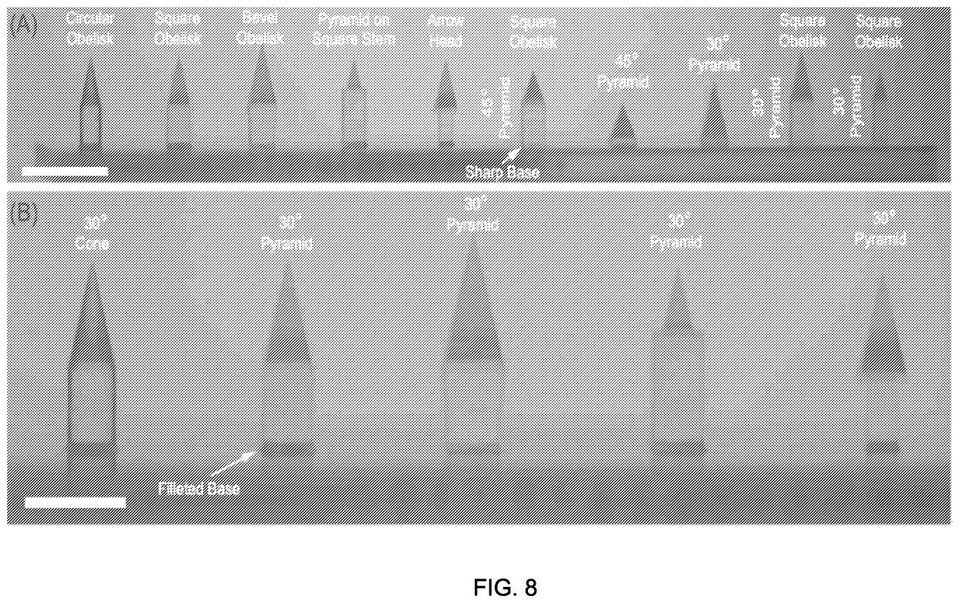

In this study, we utilized the micro-additive manufacturing process, 3D direct laser writing, to enable fabrication of complex, high accuracy, 3D microneedle geometries with smooth edges and sharp tips, as well as undercut features. This technology offers an unprecedented level of flexibility for MNA designs. To demonstrate the range of geometric capability of 3D direct laser writing, we fabricated microneedle designs with diverse geometries. This technology enabled fabrication of a wide range of microneedle geometries with high-fidelity, supporting application-driven optimization. Furthermore, as shown in FIG. 8, it allows a wide range of design changes, including height, width, apex angle, and geometry of the microneedles without requiring complex and custom processing steps. From a strength of materials standpoint, removal of MNAs with different undercut microneedle geometries is governed by needle geometry and material strength, as well as by elasticity and strength of production mold materials. Microneedles with larger undercut geometries may require more flexible production molds with lower Young's moduli for failure-free removal. More flexible PDMS molds can be prepared by adjusting the crosslinker ratio and/or curing temperature (Johnston et al., “Mechanical characterization of bulk Sylgard 184 for microfluidics and microengineering”, 2014, J. Micromech. Microeng., 24: 35017; Khanafer et al., “Effects of strain rate, mixing ratio, and stress-strain definition on the mechanical behavior of the polydimethylsiloxane (PDMS) material as related to its biological applications”, 2009, Biomed. Microdevices, 11:503-508), and hyper-elastic materials, such as Ecoflex, could serve as more flexible alternatives to PDMS (Jeong et al., “PDMS-Based Elastomer Tuned Soft, Stretchable, and Sticky for Epidermal Electronics”, 2016, Adv. Mater., 28:5830-5836), allowing removal of even larger undercut features. Collectively, 3D direct laser writing and use of flexible production molds pave the way for fabrication of a broad range of application-driven MNA designs.

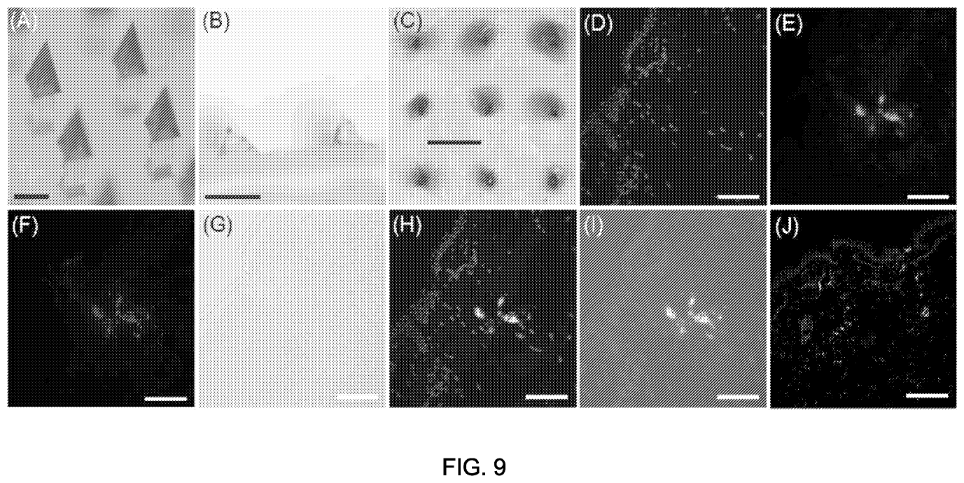

Dissolving undercut MNAs deliver multicomponent vaccines to human skin: To evaluate cutaneous biocargo delivery characteristics of dissolving MNAs with undercut microneedles, MNAs tip-loaded with Allura Red R40 dye were manufactured using the presented fabrication strategy. Allura Red R40 dye-loaded MNAs were applied to living human skin explants and removed after 10 min. Images of these MNAs before (FIG. 9 (A)) and after (FIG. 9 (B)) application demonstrated high-quality MNAs and complete dissolution of the microneedles, respectively. The corresponding deposits of MNA-embedded Allura Red R40 dye in the targeted skin are shown in FIG. 9 (C).

Successful vaccine delivery through the stratum corneum into the immune cell-rich cutaneous microenvironments is critical for effective intradermal immunization. To anatomically evaluate the delivery of antigen (OVA) and adjuvant (Poly(I:C)) into human skin, MNAs incorporating both Alexa555-labeled OVA and Alexa488-labeled Poly(I:C) were applied to human skin explants for 10 min and then removed. The targeted human skin was cryo-sectioned and imaged using epifluorescence microscopy. The resulting images demonstrated microneedle cavities penetrating through the epidermis into the dermis (FIG. 9 (G)), and delivery of fluorescent labeled OVA and Poly(I:C) to targeted human skin microenvironments (FIG. 9 (D-J)). Collectively, these results indicate that the MNAs fulfilled the geometric (sharp tips and smooth edges) and mechanical-strength requirements for failure-free human skin penetration (e.g., breaching through the stratum corneum and epidermis), and material requirements for efficient dissolution in the aqueous environment of the skin, thereby presenting an effective cutaneous drug and vaccine delivery platform.

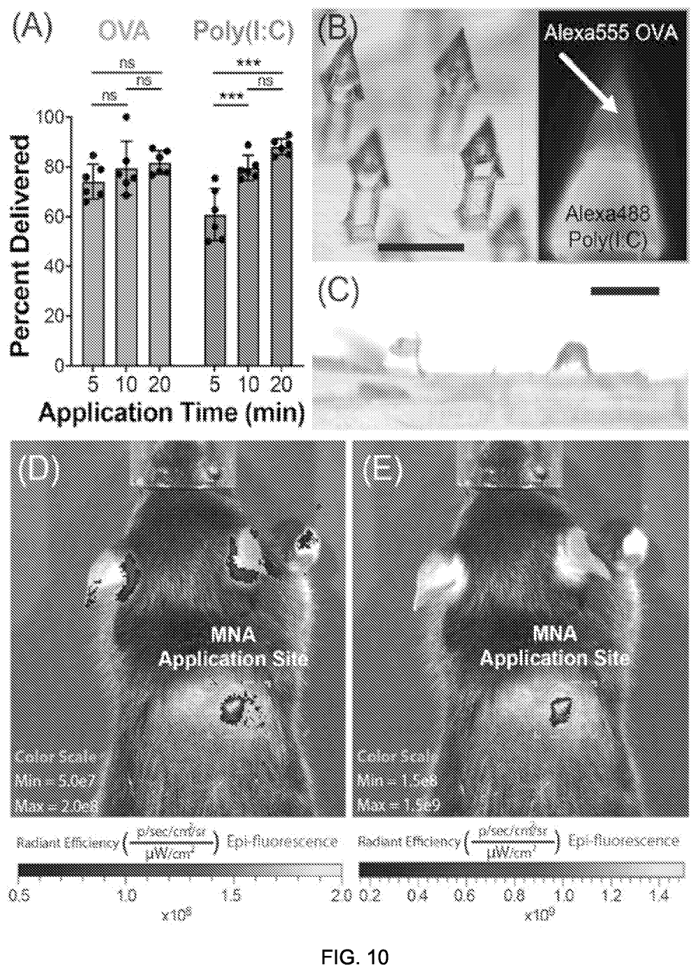

Dissolving undercut MNAs deliver multicomponent vaccines to murine skin: To evaluate cutaneous delivery efficiency and kinetics for undercut MNAs, OVA+Poly(I:C) MNAs were applied to murine abdominal skin for 5, 10, or 20 min, then removed and the remaining biocargo content was measured. Within 10 min, MNAs delivered 80.2%±12.5% OVA and 79.6±5.0% Poly(I:C), with nonsignificant additional delivery for either vaccine component by 20 min (FIG. 10 (A)). To visually confirm in vivo intradermal multicomponent vaccine delivery in mice, dissolving MNAs with high-fidelity undercut microneedles incorporating both Alexa555-OVA and Alexa488-Poly(I:C) were fabricated as described above. Prior to application, Alexa555-OVA+Alexa488-Poly(I:C) MNAs were imaged using optical stereomicroscopy and epifluorescence microscopy (FIG. 10 (B)). MNAs were then applied to mice and removed after 10 min. The remaining MNA material after applications was also imaged using optical stereomicroscopy (FIG. 10 (C)). MNA-treated mice were imaged using the IVIS 200 live animal imaging system with filters for detection of both Alexa488-Poly(I:C) and Alexa555-OVA. MNA-directed co-delivery of OVA (antigen) and Poly(I:C) (adjuvant) are shown in FIG. 10 (D, E). Together, these images demonstrate successful in vivo application of dissolvable undercut MNAs to mice and efficient delivery of both components of a multicomponent vaccine.

Multicomponent vaccine MNAs induce potent cellular and humoral immunity: Upon demonstration of successful intradermal delivery of the vaccine components to mice, we specifically evaluated immunogenicity of MNA-embedded antigen±adjuvant and compared MNA immunization to vaccination by the clinically common intramuscular (IM) injection route. To this end, MNAs were fabricated with 10 μg OVA±25 μg Poly(I:C) per MNA, as described above. We and others have previously shown that proteins integrated in dissolving MNAs maintain their integrity (Bediz et al.; Korkmaz et al.). Mice were immunized twice with MNAs or by IM injections, as detailed above, and OVA-specific cytotoxic T-cell (CTL) and antibody responses were quantified using standard in vivo lytic assay and ELISAs, respectively (FIGS. 11A-11C). Notably, dissolving MNAs with different geometries have been used to deliver antigens and other adjuvants (Zhao et al., “Enhanced immunization via dissolving microneedle array-based delivery system incorporating subunit vaccine and saponin adjuvant”, 2017, Int. J. Nanomedicine, 12: 4763-4772; Ding et al., “Microneedle arrays for the transcutaneous immunization of diphtheria and influenza in BALB/c mice”, 2009, J. Control. Release, 136:71-78; McCrudden et al., “Laser-engineered dissolving microneedle arrays for protein delivery: potential for enhanced intradermal vaccination”, 2015, J. Pharm. Pharmacol., 67:409-425), while the Poly(I:C) adjuvant used in this study has only been incorporated in coated or sustained release MNAs (Weldon et al., “Effect of adjuvants on responses to skin immunization by microneedles coated with influenza subunit vaccine”, 2012, PLoS One, 7: e41501; DeMuth et al., “Composite dissolving microneedles for coordinated control of antigen and adjuvant delivery kinetics in transcutaneous vaccination”, 2013, Adv. Funct. Mater., 23: 161-172; DeMuth et al., “Implantable silk composite microneedles for programmable vaccine release kinetics and enhanced immunogenicity in transcutaneous immunization”, 2014, Adv. Healthc. Mater., 3: 47-58).

Cutaneous vaccination with MNAs elicited robust antigen-specific cellular immune responses (FIGS. 11A and 11B). As expected, equivalent numbers of antigen-pulsed (CFSE^{high}) target cells and unpulsed (CFSE^{low}) target cells were recovered from spleens of unimmunized mice (FIG. 11A), indicating the absence of antigen-specific cytolytic activity. Mice treated with Blank MNAs (without antigen or adjuvant) also did not exhibit OVA-specific CTL responses (comparable to naïve). In contrast, specific lysis of antigen-pulsed target cells was dramatically enhanced in immunized mice, as shown by reduced survival of OVA-pulsed targets compared to unpulsed targets (FIG. 11A). While immunization by IM injections of OVA elicited a relatively low antigen-specific cellular immune response (i.e., OVA-specific lysis), cutaneous vaccination with OVA MNAs led to significantly greater OVA-specific lysis (FIGS. 11A and 11B). Importantly, addition of Poly(I:C), a Toll-like receptor 3 (TLR3) agonist adjuvant, to MNAs further improved vaccine immunogenicity, as indicated by a greater CTL response to multicomponent OVA+Poly(I:C) MNAs (FIGS. 11A and 11B). The enhanced CTL response we observed with Poly(I:C) adjuvant is consistent with previous reports that TLR3 ligands activate keratinocytes, innate immune cells, and professional APCs and induce cross-presentation of antigen to prime CD8⁺ T cells (Datta et al., “A subset of toll-like receptor ligands induces cross-presentation by bone marrow-derived dendritic cells”, 2003, J. Immunol., 170: 4102-4110; Schulz et al., “Toll-like receptor 3 promotes cross-priming to virus-infected cells”, 2005, Nature, 433:887-892; Kalali et al., “Double-stranded RNA induces an antiviral defense status in epidermal keratinocytes through TLR3-, PKR-, and MDA5/RIG-1-mediated differential signaling”, 2008, J. Immunol., 181: 2694-2704). Given these results with a model antigen, multicomponent cutaneous vaccination using dissolving undercut MNAs that incorporate pathogen- or tumor-specific antigens and adjuvants may be expected to induce robust cellular immunity essential for prevention and/or treatment of many infectious diseases and cancer (He et al., “Skin-derived dendritic cells induce potent CD8⁺ T cell immunity in recombinant lentivector-mediated genetic immunization”, 2006, Immunity, 24: 643-656; Morelli et al.; Condon et al.).

In addition to cellular immunity, cutaneous vaccination with OVA±Poly(I:C) MNAs elicited robust antigen-specific humoral immune responses (FIG. 8 (C)). While IM immunization resulted in modest OVA-specific serum IgG antibody responses, mice immunized with equivalent doses of OVA antigen in dissolving MNAs had significantly higher IgG levels. Mice not exposed to OVA antigen (i.e., naïve and Blank MNA treated mice) had undetectable levels of OVA-specific antibodies. In particular, we measured serum levels of two subclasses of OVA-specific IgG: IgG1 and IgG2c. Typically, IgG1 antibodies are associated with Th2 type immune responses to extracellular pathogens, while IgG2c antibodies are associated with Th1 type immune responses to viruses and other intracellular pathogens (Nimmerjahn et al., “Divergent immunoglobulin g subclass activity through selective fc receptor binding”, 2005, Science, 310: 1510-1512). Although both IgG subclasses can potentially neutralize and/or opsonize pathogens, IgG2c antibodies can also activate the complement pathway and typically evoke more potent cellular responses because of a greater affinity for activating Fc receptors (FcγRI, FcγRIII, and FcγRIV) and lower affinity for the inhibitory Fc receptor (FcγRIIB) (Nimmerjahn et al.). In our immunization experiments, the addition of Poly(I:C) adjuvant had minimal effect on OVA MNA induced IgG1 responses, but promoted a modest increase in IgG2c responses, consistent with the enhanced CTL immunity. Compared to IM immunization, the stronger and more balanced IgG1/IgG2c responses to cutaneous vaccination with MNAs could translate into enhanced protection against different types of pathogens. Taken together, these results demonstrate that dissolving MNAs with undercut microneedles can efficiently deliver antigens±adjuvants to APC rich microenvironments within the skin to induce potent cellular and humoral immunity. Ultimately, these undercut MNAs represent a novel modular platform technology for the specific and precise delivery of embedded multicomponent vaccines (antigen±adjuvant) to defined microenvironments within the skin.

### Conclusions

We have described a comprehensive approach to fabricate novel dissolving MNAs with undercut microneedles for effective multicomponent cutaneous vaccination. Our manufacturing approach strategically combined 3D laser lithography with nanoscale resolution and micromolding with mechanically flexible molds that allow direct removal of undercut MNAs. Reproducible fabrication of dissolvable MNAs with undercut microneedles incorporating multiple cargos was achieved using different biocompatible and water-soluble polymers, and these MNAs successfully delivered biocargos to murine and human skin microenvironments. Importantly, cutaneous vaccination with antigen-loaded MNAs elicited more potent antigen-specific cellular and humoral immune responses than traditional immunization by intramuscular injection. Simultaneous delivery of adjuvant (Poly(I:C)) to the same skin microenvironment as antigen (OVA) enhanced immune responses and may reduce the amount of antigen and/or adjuvant needed, reducing both the risk of systemic toxicity and cost. Ultimately, our approach to fabrication of dissolving MNAs with diverse geometries, including undercut microneedles, is expected to have a broad range of cutaneous and non-cutaneous vaccination and drug delivery applications.

### Example 2

Traditional vaccines, though often effective in inducing antibody responses, frequently fail to generate robust cytotoxic-T-cell (CTL) responses, which are essential to prevent or treat many cancers or infectious diseases. Currently, induction of antigen-specific cellular immunity is a point of emphasis in the vaccine field, as evidenced by recent efforts to generate “universal vaccines” for mutable infectious agents (e.g., influenza, HIV, and Coronaviruses). These target infected cells expressing functionally essential viral antigens, instead of, or in addition to, more traditional viral surface proteins that are targeted by antibodies but are highly mutable. Successful integration of adenovector vaccines onto coated, or into dissolvable, MNAs can induce efficacious and durable antigen-specific responses. Despite promising results, clinical translation of adenovector vaccines has been hampered by limited efficacy, thereby defining an unmet need to enhance the immune responses induced by adenovirus vaccines.

Here, we generated a three-dimensional (3D) multicomponent skin-targeted vaccine platform, combining an adenovirus-encoded antigen with an adjuvant to induce stronger cellular immune responses. Specifically, we developed dissolving MNAs to simultaneously co-deliver adenovectors encoding a transgene expressing an antigen, together with an adjuvant. As shown in this example, the transgene for the model antigen ovalbumin (OVA), together with Poly(I:C), a Toll-like receptor 3 (TLR3)-triggering double-stranded RNA molecule, are delivered to the cutaneous microenvironment, with the primary goal of enhancing antigen-specific cellular immune responses. Dissolvable MNAs are designed to mechanically penetrate the superficial cutaneous layers, rapidly dissolve upon insertion into the skin, and deliver uniform quantities of biocargo to a defined 3D space within the skin. They enable localized delivery of low amounts of drugs or vaccines to achieve high concentrations in a specific skin microenvironment.

Innate cell-signaling pathways (e.g., downstream of TLRs) are well-studied targets for the rational design of vaccine adjuvants for protein subunit vaccines; however, they have yet to be comprehensively evaluated in the context of recombinant viral vectored vaccines due to significant mechanistic differences, including differences in the kinetics and amount of antigen expression. Among TLR family members, TLR3 signaling imparts unique responses, such as secretion of immunostimulatory IFN-β and CXCL10, due to its distinct downstream pathways. While other TLRs signal through adaptor protein MyD88, TLR3 uses the TIR-domain containing adapter-inducing interferon-β (TRIF) adapter protein, with subsequent activation of IRF3 and IRF7. Here, using in vivo mouse models, we demonstrate that MNA delivery of the TLR3 agonist Poly(I:C) with antigen-encoding adenovectors results in proinflammatory changes in the targeted skin microenvironment that correlate with robust antigen-specific cellular and humoral adaptive immune responses.

### Materials and Methods

### Fabrication of Microneedle Arrays

Dissolving MNAs with obelisk-shaped needles that incorporate adenovectors with or without Poly(I:C) were manufactured using our previously described MNA fabrication strategy (Korkmaz E, et al., Therapeutic intradermal delivery of tumor necrosis factor-alpha antibodies using tip-loaded dissolvable microneedle arrays. Acta Biomater. 2015 September; 24:96-105). Our MNAs are designed for human applications and are currently being used in a Phase I clinical trial for treatment of cutaneous T cell lymphoma (ClinicalTrials.gov #NCT02192021). Our fabrication methods are flexible to rapidly modify the microneedle and array designs for application-driven optimization (Bediz B, Korkmaz E, Khilwani R, Donahue C, Erdos G, Falo L D, et al. Dissolvable microneedle arrays for intradermal delivery of biologics: Fabrication and application. Pharm Res 2014; 31(1):117-135 and Balmert S C, Carey D C, Falo G D, Sethi S K, Erdos G, Korkmaz E, Falo L D, Jr. Dissolving undercut microneedle arrays for multicomponent cutaneous vaccination. J Control Release 2020; 317: 336-346). Briefly, MNA production molds were prepared using polydimethylsiloxane (PDMS, SYLGARD® 184 from Dow Corning, 10:1 base material to curing agent ratio) through elastomer micromolding with MNA master molds which include 750 μm high microneedles in a 10×10 array configuration. We previously demonstrated that the same MNAs can deliver cargos to antigen presenting cell rich skin microenvironments in both mice and humans (Bediz B, et al., Pharm Res 2014; 31(1):117-135). Next, PDMS production molds were used to fabricate dissolvable MNAs integrating Ad5.OVA (2×10⁹ genome count per MNA)±Poly(I:C) (100 μg per MNA) via a multi-step spin-casting technique. Sequential loading of Poly(I:C) and Ad5.OVA was performed via centrifugation at 4° C. and 3500 rpm for 1 hr for each loading. After loading biocargos, the structural material of MNAs, prepared by dissolving carboxymethyl cellulose (CMC, cat #C5678, Sigma Aldrich, St Louis, Mo.) and trehalose (cat #T9531, Sigma-Aldrich, St Louis, Mo.) powders in endotoxin-free water (HyClone Cell Culture Grade Water) at 15% w/wand 10% w/w, respectively, resulting in 25% w/w final solute concentration, was loaded onto PDMS production molds (75 μL of CMC/Treh hydrogel per MNA) and centrifuged at 10° C. and at 3500 rpm for 6 hr. Furthermore, blank MNAs without any biocargo were prepared from the same material composition for control experiments. Fabricated MNAs were imaged with a bright field stereo microscope.

### Fluorescent Labeling

Adenovirus and Poly(I:C) were labeled using Alexa Fluor 555 and 488 fluorescent dyes, respectively. To label viral capsids, amine-reactive Alexa 555 dye (cat #A20009, ThermoFisher) was used according to manufacturer's instructions with a minor modification, which is the direct solubilization of Alexa 555 dye in the viral suspension, avoiding the use of dimethylformamide (DMF) that may impart detrimental effects on the capsid structure. To label Poly(I:C), its amine modification was performed as previously described (Hermanson et al., 1996). Briefly, 5 mg/ml Poly(I:C) was denatured at 95° C. for 5 min and reacted to 3 M ethylenediamine in the presence of 1 M sodium bisulfite at 42° C. for 3 hr. The reaction mix was dialyzed overnight at 4° C. The resultant aminated Poly(I:C) was ethanol precipitated, air dried, and resuspended in water. Finally, amine-reactive Alexa-488 NHS ester (cat #: A2000, ThermoFisher) was used to label NH2-Poly(I:C) conjugate according to manufacturer's instructions.

Mice and Animal Husbandry

C57BL/6J mice were purchased from The Jackson Laboratory (Bar Harbor, Me.), maintained under specific pathogen-free conditions, and used at 8-10 weeks of age.

In Vivo Imaging

MNA-mediated skin-targeted co-delivery of Ad5.OVA and Poly(I:C) was demonstrated on a C57BL/6J mouse. MNAs integrating Alexa555-Ad5.OVA and Alexa488-Poly(I:C) were created as described above, applied to the ear of an anesthetized mouse for 10 min, and then removed. The mouse was imaged with an in vivo live animal imaging system (IVIS 200, PerkinElmer, Waltham, Mass.) to detect Alexa488-Poly(I:C) and Alexa555-Ad5.OVA at the MNA application site. Images were then post-processed using Living Image software (PerkinElmer).

Quantification of CTL and Antibody Responses

Antigen (OVA)-specific cell-mediated immunity was determined by evaluating OVA-specific cell lysis in groups of four female C57BL/6J mice, immunized by ear applications of Ad.OVA-MNAs, Ad.OVA+Poly(I:C)-MNAs or blank MNA (control). Twelve days after immunization, mice were assayed for OVA-specific T-cell lytic activity using well-established techniques. Briefly, splenocytes from naïve mice were pulsed with 2 μg/ml OVA derived SIINFEKL (SEQ ID NO: 1) peptide epitope or left unpulsed for 1 h. The unpulsed splenocytes were labeled with low concentration GFSE (1 μM) for 15 min at 37° C., while the antigen pulsed splenocytes were washed and stained with high concentration GFSE (10 μM). Equal populations of the pulsed and unpulsed target cells (2×10⁷ splenocytes per mouse) were injected intravenously (IV) into immunized and naïve mice. Twenty hours after the injection, the spleens were recovered from all the animals, and the killing of target cells was evaluated by comparison of the antigen pulsed and unpulsed populations by flow cytometry to quantify antigen specific killing of the high GFSE labeled SIINFEKL (SEQ ID NO: 1)-pulsed targets. Specific lysis was calculated according to the following formula: {1−[(ratio of CFSElow/CFSEhigh of naïve mouse)/(ratio of CFSElow/CFSEhigh of vaccinated mouse)]}×100, and expressed as % of maximum lysis.

Antigen (OVA)-specific antibody responses were determined in groups of four female C57BL/6J mice, immunized by ear applications of Ad.OVA-MNAs, Ad.OVA+Poly(I:C)-MNAs or blank MNA (control). Thirty days after immunization, the standard curves for OVA-specific IgG1 and IgG2 antibodies were obtained, blood was collected from anesthetized mice at the time of sacrifice by cardiac puncture, serum was isolated using BD Microtainer serum separator tubes (BD Biosciences, San Jose, Calif.) and diluted to the levels within the standard curves to quantify OVA-specific IgG1 and IgG2c antibodies in serum by indirect ELISAs as previously described (Balmert S C, et al., J Control Release 2020; 317: 336-346).

Intercutaneous immunization experiments were repeated with Ad.OVA+Poly(I:C) loaded MNAs stored at 4° C. for one month and the associated quantification of cell-mediated and humoral immune responses was again performed as described above.

### Real-Time Quantitative RT-PCR

MNA-treated ear tissue was recovered after 6, 24, 48, and 72 hr and analyzed for specific gene expression of immune mediators by qRT-PCR. Skin was homogenized at 4° C. in TRI-reagent (Molecular Research Center, Cincinnati, Ohio) using a Bullet Blender Storm 24 with stainless steel beads in Navy RINO tubes (Next Advance, Averill Park, N.Y.). Total RNA was extracted according to the manufacturer's protocol and quantified using a DeNovix DS-11 spectrophotometer (Wilmington, Del.). For each reverse transcription reaction, 2 μg RNA was converted to cDNA using a QuantiTect Reverse Transcription Kit (Qiagen, Germantown, Md.). Quantitative real-time PCR was then performed using TaqMan Fast Advanced Master Mix (Thermo Fisher), according to manufacturer's instructions, with TaqMan Gene Expression assays (Applied Biosystems, Carlsbad, Calif.) specific for IFN-β1 (Mm00439552_s1), Cxcl10 (Mm00445235_m1), IL-1β (Mm00434228_m1), and IL-6 (Mm00446190_m1). Target gene primer-probe assays were FAMMGB labeled, while the ACTB endogenous control primer-probe assay (Mm00607939_s1) was VIC-MGB_PL labeled. Duplex reactions (target gene+ACTB) were analyzed on a StepOnePlus Real-Time PCR System (Applied Biosystems). Expression of each target gene was calculated and normalized to the ACTB endogenous control and naïve ear skin based on the Livak (2^{-ΔΔCt}) method. The relative quantities were expressed as fold differences relative to naïve skin.

### Statistical Analysis

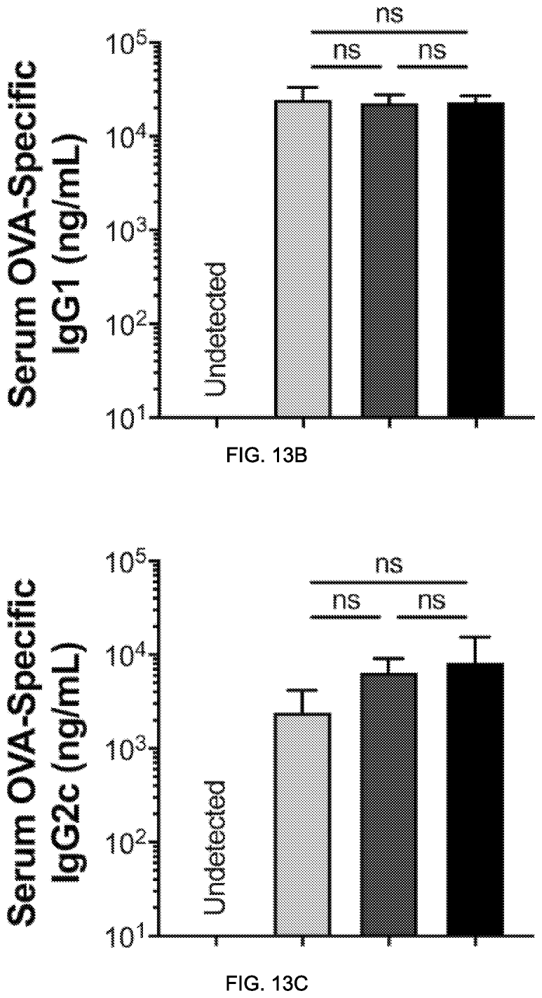

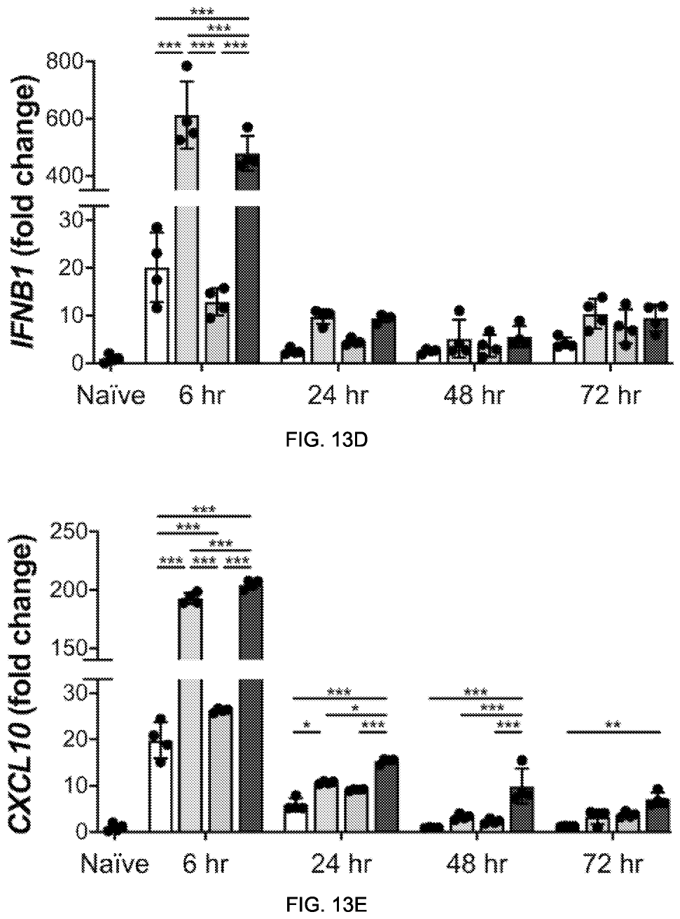

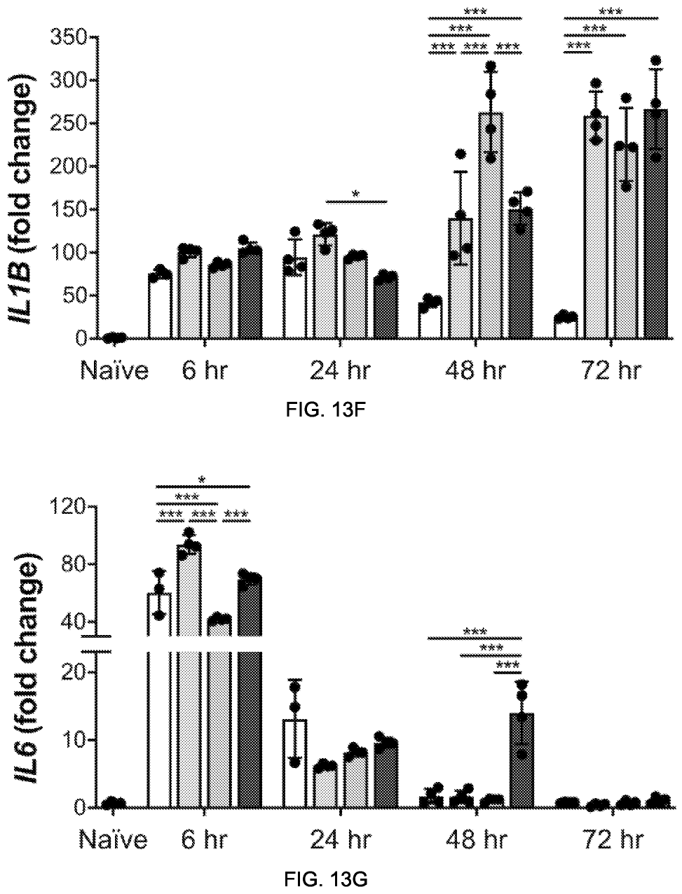

GraphPad Prism v8 (San Diego, Calif.) software was used for statistical analyses. Data were represented as mean value±standard deviation and analyzed by either one-way ANOVA, followed by Tukey's post-hoc tests (FIGS. 13A-13C) or two-way ANOVA, followed by either Sidak's (FIG. 12F) or Tukey's (FIGS. 13D-13G) multiple comparisons test. ns>0.05, * p<0.05, ** p<0.01, *** p<0.001, **** p<0.0001.

### Results

For these studies, mice were used at 8-10 weeks of age. Dissolvable MNAs incorporating adenovirus vaccines (Ad5.OVA) with or without Poly(I:C) were manufactured using a composition of two GRAS water-soluble biomaterials: carboxymethyl cellulose and trehalose. Optical-microscopy images of MNAs before and after in vivo application to skin demonstrated high-quality micron-scale needles after fabrication and effective dissolution upon skin insertion, respectively (FIGS. 12A and 12C). Importantly, the multicomponent MNAs effectively delivered adenovirus (FIG. 1C) and Poly(I:C) (FIG. 12D) to the skin microenvironment in vivo (FIG. 12E), resulting in transgene (OVA) expression (FIG. 12F). Interestingly, compared to MNA delivery of adenovector alone, inclusion of Poly(I:C) was associated with significantly (p<0.0001) increased expression of OVA mRNA that was sustained through 48 h.

Intercutaneous vaccination with MNAs generated robust antigen-specific cytotoxic and humoral immune responses. Remarkably, multicomponent MNA vaccine platforms incorporating both antigen-encoding adenovector and Poly(I:C) augmented OVA-specific lytic immunity by approximately two-fold compared to MNA-delivery of the same adenovector alone (FIG. 13A). In addition to cell-mediated immunity, MNA adenovirus vaccine platforms, with or without the addition of Poly(I:C), elicited strong and robust antigen-specific antibody responses (IgG1 and IgG2c) (FIGS. 13B and 13C). Thus, adding Poly(I:C) to this MNA-delivered adenovirus vaccine significantly improved antigen-specific cellular immunity while maintaining strong antibody responses. Notably, multicomponent MNAs integrating both Poly(I:C) and adenovirus retained their immunogenicity after one-month of storage at 4° C., as indicated by no statistically significant loss in cell-mediated or antibody responses (FIGS. 13A and 13C).

Mechanistically, simultaneous co-delivery of Poly(I:C) with adenovector vaccines impacted the pro-inflammatory microenvironment at the immunization site (FIG. 13D-13G). In particular, statistical analyses showed that the addition of Poly(I:C) significantly increased IFNB1 (FIG. 13D) and CXCL10 (FIG. 13E) expression at 6 hr, with respect to blank (empty) MNAs or MNAAd alone, which suggests that Poly(I:C) plays a distinctive role during early skin immunomodulation. Furthermore, the inclusion of Poly(I:C) continued to significantly enhance the expression of CXCL10 (FIG. 132E) at later time points (24 hr and 48 hr) compared to blank MNA and MNA-Ad alone groups, consistent with a sustained chemoattractant effect of Poly(I:C). Importantly, these pro-inflammatory effects of Poly(I:C) correlate with enhanced systemic CTL responses. Expression of the proinflammatory cytokines IL-1 and IL-6, which can be induced by a broad range of pathogen associated molecular patterns (PAMPS) and danger associated molecular patterns (DAMPs), was elevated in MNA-immunized skin microenvironments at early time points (6 hr) regardless of vaccine components (FIGS. 13F and 13G), likely as a result of the mechanical stress of microneedle application. Interestingly, at later time points (48 hr and 72 hr), after resolution of the transient mechanical stress generated by microneedles, both Adenovirus and/or Poly (I:C) evoked significant increases in IL-1B expression in the skin microenvironment, and the combination of Ad.OVA and Poly(I:C) sustained elevated levels of IL-6 through 48 h.

Collectively, our results demonstrate improved immunogenicity of skin-targeted adenovector vaccines by simultaneous co-delivery of the TLR3 ligand Poly(I:C), and support further development of PAMP/DAMP ligand integration in MNA-delivered viral vector vaccines. Specifically, our results demonstrate that Poly(I:C) adjuvanted MNA adenovirus vaccines elicit significantly improved CTL responses compared to adenovirus alone, while generating antibody responses at least as good as adenovirus alone. MNA-delivered vaccines have the potential to offer advantages of ease of fabrication, application, and storage compared to other vaccine delivery platforms. Our results suggest that multicomponent MNA vaccine platforms, uniquely enabling delivery of both adjuvant and antigen-encoding viral vectors to the same skin microenvironment, result in improved immunogenicity including cellular immune responses, thereby contributing to efforts to develop universal vaccines and improve global immunization capabilities.

Having described this invention, it will be understood to those of ordinary skill in the art that the same can be performed within a wide and equivalent range of conditions, formulations and other parameters without affecting the scope of the invention or any embodiment thereof.

1. A microneedle device, comprising:
a backing layer; and
a plurality of microneedles extending from the backing layer, and comprising a dissolvable (aqueous-soluble) and/or bioerodible matrix comprising trehalose, and a recombinant adenovirus particle comprising a gene for expressing a polypeptide or RNA.

2. The microneedle device of claim 1, wherein the recombinant adenovirus in the microneedle device retains viability for at least one month at 4° C.

3. The microneedle device of claim 1, wherein the number of infectious units (IU) of the recombinant adenovirus in the microneedle decreases by less than 50%, 40%, 30%, or 25% on storage for one month at 4° C.

4. The microneedle device of claim 1, wherein the gene expresses an immunogen.

5. (canceled)

6. The microneedle device of claim 5, wherein the microneedle further comprise a compound or composition having immune stimulant or adjuvant effect.

7. The microneedle device of claim 6, wherein the compound or composition having immune stimulant or adjuvant effect is a double-stranded RNA, or an analog or derivative thereof; a TLR3 agonist′ a TLR4 agonist; a TLR5 agonist; and TLF 7/8 agonist; a TLR9 agonist; a Stimulator of Interferon Genes (STING) pathway agonist; a stimulatory neuroimmune mediator; a neurokinin 1 (NK1) receptor agonist; a saponin related adjuvant; a purinoergic receptor agonist; an oil-in-water emulsion adjuvant; an LPS, monophosphoryl lipid derivative; a flagellin derivative; imiquimod; R848, a CpG nucleic acid sequence; ADU-S100; a calcitonin gene-related peptide (CGRP); Hemokinin 1; Substance P; QS-21 (Quillaja saponaria); ATP; or MF59.

8. (canceled)

9. The microneedle device of claim 6, wherein the compound or composition having immune stimulant or adjuvant effect is Poly(I:C) or Poly-ICLC.

10. (canceled)

11. (canceled)

12. The microneedle device of claim 1, wherein the gene expresses an antisense RNA or an RNAi reagent.

13. (canceled)

14. The microneedle device of claim 1, wherein the plurality of microneedles have a shape that comprises a first cross-sectional dimension at a head portion distal to the backing layer, a second cross-sectional dimension at a stem portion proximal to the backing layer, and a third cross-sectional dimension at an intermediate portion located between the top portion and the bottom portion having a cross-sectional dimension greater than the first and second cross-sectional dimensions.

15. The microneedle device of claim 14, wherein the plurality of microneedles comprise a stem portion adjacent or proximal to the backing layer, and a head portion attached to the stem portion distal to the backing layer, the microneedles having a barbed or undercut profile in which the head portion having a cross section adjacent to the stem is larger than a cross section of the stem adjacent to the head.

16. The microneedle device of claim 14, wherein a plurality of microneedles comprise a plurality of layers of one or more dissolvable or bioerodible, biocompatible material.

17. The microneedle device of claim 1, wherein the plurality of microneedles further comprise carboxymethyl cellulose, polyvinylpyrrolidone, maltodextrin, silk, hyaluronic acid, poly(lactic-co-glycolic acid), poly(lactic acid), poly(vinyl alcohol), polyethylene glycol, or a combination of any two or more of the preceding.

18. (canceled)

19. The microneedle device of claim 1, wherein the plurality microneedles have a layered structure with the adenovirus particles located substantially at the tip of the microneedles.

20. The microneedle device of claim 1, wherein the plurality of microneedles each have a length of 1 mm or less.

21. The microneedle device of claim 1, wherein the plurality of microneedles have a filleted base.

22. The microneedle device of claim 1, wherein the plurality of microneedles comprise a stem, a head, a filleted base, and at least one undercut feature.

23. The microneedle device of claim 22, wherein the plurality of microneedles comprise the recombinant adenovirus particles located substantially in the head of the microneedles.

24. The microneedle device of claim 22, wherein the at least one undercut feature is directly below the microneedle head.

25. The microneedle device of claim 22, wherein the stem is comprises a dissolvable or bioerodible material that optionally rapidly dissolves in water, e.g. dissolves in less than 30 seconds, less than 20 seconds, less than 10 seconds, or less than 5 seconds in water.

26. The microneedle device of claim 25, wherein the dissolvable or bioerodible material of the stem comprises carboxymethyl cellulose, trehalose, polyvinylpyrrolidone, maltodextrin, silk, hyaluronic acid, poly(lactic-co-glycolic acid), poly(lactic acid), poly(vinyl alcohol), polyethylene glycol, glucose, sucrose, maltodextrin, polyvinylpyrrolidone, or a combination of any two or more of the preceding.

27-31. (canceled)

32. The microneedle device of claim 1, wherein the Adenovirus is an Ad5 vector for expression of a transgene.

33. A method of eliciting a therapeutic effect or expressing a transgene, in a patient, comprising placing the microneedle device of claim 1, on the skin of the patient to cause the plurality of microneedles to enter the skin of the patient, thereby the adenovirus into a cell of the patient for expression of the gene encoded by the adenovirus and eliciting the therapeutic effect in the patient.

34. (canceled)

35. A method of forming a microneedle device, comprising:
forming or providing a production mold of a flexible material, the production mold comprising a plurality of cavities that are shaped to define a plurality of respective microneedles having a stem, a head, a filleted base, and at least one undercut feature, the microneedles optionally having a length of 1 mm or less;
delivering a first dissolvable or bioerodible material comprising trehalose into at least the microneedle head portion defined by the respective cavities of the production mold, and prior to or during delivery of the first dissolvable or bioerodible material into at least the microneedle head portion, incorporating a recombinant adenovirus particle comprising a gene for expressing a polypeptide or RNA into the first dissolvable or bioerodible material to produce a dissolvable or biodegradable matrix;
delivering the first dissolvable or bioerodible material and/or one or more additional dissolvable or bioerodible materials into the cavity and forming a plurality of microneedles in the production mold that include the dissolvable or biodegradable matrix; and
removing the microneedles from the production mold by pulling the microneedles out of the mold,
wherein the flexible material of the production mold has sufficient elasticity to allow for the molded microneedle array to be removed from the production mold, e.g., in a single pull, without damaging the integrity of the shape of the microneedles as defined by the mold.

36-44. (canceled)
